(19) United States
(12) Patent Application Publication
(10) Pub. No.: US 2016/0038411 A1
(43) Pub. Date: Feb. 11, 2016

(54) PULSATILE GASTRIC RETENTIVE DOSAGE FORMS

(71) Applicant: Depomed, Inc., Newark, CA (US)

(72) Inventors: Verne Earle Cowles, Dublin, CA (US); Sui Yuen Eddie Hou, Foster City, CA (US); Bret Berner, Half Moon Bay, CA (US); Chien-Hsuan Han, Sunnyvale, CA (US); Ryan Douglas Fell, San Carlos, CA (US); Chunhong Gu, Mountain View, CA (US)

(21) Appl. No.: 14/838,031

(22) Filed: Aug. 27, 2015

Related U.S. Application Data
(62) Division of application No. 12/220,747, filed on Jul. 25, 2008, now abandoned.
(60) Provisional application No. 60/952,501, filed on Jul. 27, 2007.
(60) Provisional application No. 60/967,717, filed on Sep. 5, 2007.

(51) Int. Cl. A61K 9/00; A61K 9/28; A61K 9/48; A61K 31/4025

(52) U.S. Cl. CPC A61K 9/0065; A61K 31/4025; A61K 9/2853; A61K 9/4808; A61K 9/282; A61K 9/4866; A61K 9/4858

(57) ABSTRACT

Dosage forms for delayed and pulsed release of therapeutic agents into the stomach are described. The dosage forms are gastric retentive dosage forms that achieve release of the therapeutic agent into the stomach and upper gastrointestinal tract subsequent to administration of the dosage form. The dosage forms find particular use in administration of acid-labile active agents such as proton pump inhibitors, and in treating gastric acid secretion such as gastro-esophageal reflux disease (GERD) and nocturnal acid breakthrough (NAB).

### CROSS REFERENCE TO RELATED APPLICATIONS

This application claims the benefit of U.S. provisional application Ser. No. 60/952,501, filed Jul. 27, 2007 and of U.S. provisional application Ser. No. 60/967,717, filed Sep. 5, 2007. Both applications are incorporated by reference herein in their entirety.

### TECHNICAL FIELD

This subject matter relates generally to gastric retentive dosage forms that deliver a therapeutic agent to the stomach or upper gastrointestinal tract in one or more pulses, wherein one or both of the pulses are delivered at a time removed from ingestion of the dosage form. More particularly, the subject matter relates to gastric retentive dosage forms that deliver a drug in a first pulsed release and a second pulsed release, where at least the second pulsed release occurs at a time removed from ingestion of the dosage form, to provide two burst releases of drug into the stomach or upper gastrointestinal tract.

### BACKGROUND

Drug efficacy generally depends upon the ability of the drug to reach its target or site of action in sufficient quantity to achieve the desired therapeutic level at the desired time and to maintain the desired therapeutic level for the desired time period. A variety of dosage forms have been developed to optimize the therapeutic effect of a drug. The optimal dosage form for a particular drug is selected or designed based on a variety of factors, such as the drug's bioavailability, extent and mechanism of metabolism, and site of absorption. Oral dosage forms that provide immediate release of a drug, that is where the drug is released from the dosage form immediately or very soon after ingestion are a common approach for drug delivery. Extended or sustained release dosage forms where release of drug from the dosage form begins soon after ingestion and continues over an extended period of time are also a common approach. Delayed release dosage forms, where a drug is released from the dosage form after a period of time has elapsed after ingestion, find use for drugs or conditions that benefit drug release in the lower gastrointestinal (GI) tract.

Orally administered drugs enter the general circulation of the human body after ingestion by absorption of the drug into the capillaries and veins of the upper GI tract and transport by the portal vein to the liver. Absorption is limited, for some drugs, by the low pH and enzymatic activities in the gastric fluid, which can inactivate certain drugs, negatively affect release of the drug from the dosage form, or hinder absorption of the drug once released. Enteric coatings offer a solution to this problem, provided the coating is sufficiently acid resistant to protect the encapsulated drug until it passes into the more basic environment of the small intestine, where the coating is degraded, the drug is released, and then absorbed into the small intestine.

For drugs that are preferentially absorbed in the upper GI tract or proximal regions of the small intestine, including, for example, proton pump inhibitors (PPIs) and H₂-receptor antagonists, there is an additional obstacle in delivering an effective dose to the patient at a time removed from the time of ingestion of the drug. For such drugs, if the dosage form is not retained in the upper GI tract, then release of the drug from the dosage form at a time removed from the time of ingestion is likely to occur in the lower GI tract, where it will have limited or no therapeutic effect.

Following absorption of an orally administered drug by the digestive system, it enters the hepatic portal system. It is carried through the portal vein into the liver before it reaches the rest of the body. The liver and the wall of the intestine metabolize many drugs, sometimes to an extent such that only a small amount of active drug emerges from the liver into the rest of the circulatory system. This initial pass through the liver and the wall of the intestine is referred to in the medical arts as the first-pass effect, or as first-pass metabolism. Orally administered drugs subject to first-pass metabolism in the liver or intestinal wall and are excreted into bile or converted into pharmacologically inactive metabolites that provide no therapeutic benefit. Such drugs therefore have decreased bioavailability, relative to drugs not subject to the first-pass effect, because less of the drug administered reaches the site of drug action. The first-pass effect can be overcome by administering the drug so that it is released from the dosage form in sufficient quantities to exceed the metabolic capability of the liver. This results in nonlinear pharmacokinetics, because initially, the amount of the drug in the general circulation is lower than the amount that would result from administration in the absence of a first-pass effect. Moreover, first-pass metabolism results in variable drug absorption with the polymorphic forms of the hepatic enzymes in different individuals and populations. Once the liver's metabolic capacity has been exceeded, there is a significant and abrupt increase in the drug concentration in the bloodstream.

The first-pass effect makes the sustained release of a drug preferentially absorbed in the upper GI tract highly problematic. First, sustained release of the amount of drug needed to overcome the first-pass effect may simply require too much drug or variable absorption of drug and result in blood levels that cause unwanted side effects. Second, even if the first problem can be overcome, the dosage form may pass through the digestive tract too quickly for the drug to be released in the upper GI tract where it is preferentially absorbed. Moreover, with traditional oral extended-release dosage formulations, which exhibit continuous release profiles such as those with first order or square-root of time release rates, the amount of active agent released from the dosage form diminishes as time progresses after administration. The first-pass effect can eliminate any therapeutic effect of the drug as the drug levels decrease. Although a bolus or burst delivery of the active agent could overcome the first-pass effect, there are no effective dosage forms that can deliver such a bolus or burst at a time significantly removed from the time of ingestion of the dosage form while maintaining the dosage form in the upper GI tract.

Drug delivery systems developed for orally administered drugs subject to the first-pass effect include formulations capable of immediate drug release that are suitable for administration from 3-4 times daily, and formulations capable of immediate and sustained drug release that are suitable for once-daily administration. The second type of formulation is preferred, because patient compliance with prescribed drug regimens involving once-daily administration is substantially greater than those involving more than once daily administrations. There remains a need for new dosage forms that can be used to administer drugs subject to the first-pass effect that are preferentially absorbed in the upper GI tract.

For example, gastro-esophageal reflux disease (GERD) is a disease in which stomach acid reflux, or back flow from the stomach into the esophagus. GERD is treated with drugs preferentially absorbed in the upper small intestine and subject to the first-pass effect. GERD is a common disease, present in approximately 40% of adults in the United States on an intermittent basis and some 10% on a daily basis (see U.S. Pat. No. 6,098,629 to Johnson et al., incorporated herein by reference). GERD is characterized by the abnormal and prolonged exposure of the esophageal lumen to acidic gastric contents (Hunt, Ailment Pharmacol Ther. 9(Supp. 1):37 (1995)). Many factors are believed to contribute to the onset of GERD, including transient lower esophageal sphincter relaxations, decreased lower esophageal sphincter resting tone, delayed stomach emptying, and an ineffective esophageal clearance.

A common symptom of GERD is heartburn, a burning sensation or discomfort behind the breastbone or sternum. Other symptoms of GERD include dysphasia, odynophagia, hemorrhage, water brash, and pulmonary manifestations such as asthma, coughing, or intermittent wheezing due to acid aspiration. Patients suffering from GERD commonly suffer from these symptoms at mealtimes and at bedtime. A condition experienced by many GERD patients is nocturnal acid breakthrough or “NAB” (Peghini et al., Am. J. Gastroenterol. 93:763-767 (1998)), because gastric acid secretion varies throughout the day and may be most pronounced at night. A surge of gastric acidity is common around 2 A.M.

Control of GERD can include lifestyle changes, such as weight loss, avoidance of certain foods and excessive bending, and elevation of the head of a patient's bed to prevent nocturnal reflux, and surgery (e.g., fundoplication, Collis-Nissen gastroplasty, bulking the lower esophageal sphincter, restricting the esophagus, and obesity treatments); drug therapy is often the treatment of choice.

Drugs used to treat GERD include H₂-receptor antagonists (which control gastric acid secretion in the basal state) and PPIs (which control both basal and meal-stimulated acid secretion). Both classes of drugs can raise intragastric pH to greater than about 4 for varying durations. The PPI class of drugs can permanently shut down all proton pumps active at the time a PPI is administered, but inactive proton pumps remain unaffected, and new proton pumps are continuously created (especially during the night-time hours). GERD patients on PPI therapy therefore suffer GERD symptoms during the night, especially as PPIs are administered at mealtimes or once daily in the morning.

Omeprazole (5-methoxy-2-[[(4-methoxy-3,5-dimethyl-2-pyridinyl)methyl]sulfinyl]-1H-benzimidazole; see U.S. Pat. No. 5,877,192 to Lindberg et al.) is a PPI and may also be referred to as an H⁺K⁺-ATPase inhibitor. Other PPIs include lansoprazole, pantoprazole, pariprazole, rabeprazole, esomeprazole, tenatoprazole, and leminoprazole. These compounds are generally effective as gastric acid secretion inhibitors but are acid labile, subject to the first-pass effect, and preferentially absorbed in the small intestine. Omeprazole and other PPIs have absorption characteristics that render controlled-release delivery problematic. Because PPIs are unstable in acid, efficacious delivery typically requires an enteric coating around the drug for protection from the acidic environment of the stomach or a base in the drug formulation to protect the drug. Omeprazole may require protection even from the acidity of certain enteric coatings; such protection is typically provided with a sub-coat layer. In addition, omeprazole suffers from significant first-pass metabolism and is typically administered once daily, 30-60 minutes before a meal, usually the breakfast meal.

There remains a need for dosage forms that administer drugs susceptible to first-pass metabolism, that are degraded by the acidic conditions of the stomach, and that are preferentially absorbed in the small intestine. In addition, there remains a need for dosage forms and methods of treating GERD and of treating GERD in such a way to reduce, prevent or eliminate the occurrence of NAB.

### SUMMARY OF THE DISCLOSURE

In a first aspect, a dosage form comprising a first dose of drug that is released from the dosage form substantially immediately after oral administration, and a second dose of drug that is released from the dosage form substantially after oral administration is provided. The second dose of drug is contained in a delivery vehicle that swells by imbibing water present in gastric fluid to a size sufficient to achieve retention in a stomach in a fed mode for release of substantially all of the second dose. In one embodiment, the delivery vehicle comprises a component that protects at least a portion of the second dose from inactivation by exposure to acidic conditions in the stomach.

In one embodiment, the first dose of drug is released from the dosage form in less than about 60 minutes after ingestion of the dosage form. In another embodiment, the second dose of drug is released from the dosage form 2-6 hours after ingestion of the dosage form.

In one embodiment, the delivery vehicle is comprised of a hydrophilic polymer that swells unrestrained dimensionally in water.

In yet another embodiment, the delivery vehicle is comprised of a plurality of beads dispersed in a hydrophilic polymer that swells unrestrained dimensionally in water, each bead comprised of (a) a core; (b) drug disposed on an external surface of the core; (c) an optional coating disposed on the drug; and (d) an optional enteric coating as a component that protects at least a portion of the second dose from inactivation, wherein the plurality of beads comprise an amount of drug sufficient to provide the second dose of drug.

In still another embodiment, the delivery vehicle is comprised of a polymeric insert having a central cavity, the insert comprised of a hydrophilic polymer that swells unrestrained dimensionally in water, and the cavity comprising the second dose of drug.

In another embodiment, a plurality of beads comprise an amount of drug sufficient to provide the second dose of drug, and wherein each bead is comprised of (a) a core; (b) drug disposed on an external surface of the core; (c) an optional sub-coating disposed on the drug; and (d) an optional enteric coating as the component that protects at least a portion of the second dose from inactivation.

In yet another embodiment, the dosage form comprises a second polymeric insert, where the second insert comprises a cavity that comprises the first dose of drug.

In a preferred embodiment, the first and second inserts are contained within a capsule, and wherein an end of the first insert engages an opening of the second insert, and swelling of the inserts after oral administration creates in situ a seal between the first insert end and the second insert opening to delay release of the plurality of beads contained in the second insert.

In still another embodiment, the delivery vehicle comprising the second dose of drug is comprised of a drug core encased by the component that protects the second dose, which is surrounded by a hydrophilic polymer that swells unrestrained dimensionally in water.

The drug core, in another embodiment, comprises the drug and at least one excipient, and wherein the component that protects the second dose is an enteric coating layer disposed on the tablet core; and wherein the hydrophilic polymer forms a layer disposed on the enteric coating layer, and wherein the first dose is contained in an immediate release component disposed on the hydrophilic polymer layer.

In yet another embodiment, the delivery vehicle is comprised of (a) a tablet core comprising a plurality of beads and a matrix, wherein the beads comprise the second dose of drug; and (b) a gastric retentive layer disposed on the tablet core.

In any of the embodiments described above, the component that protects the second dose can be selected from a basic compound and an enteric coating.

In any of the embodiments described above, the first dose of drug and the second dose of drug can be same drug or different drugs. In a preferred embodiment, both doses are a proton pump inhibitor. A preferred proton pump inhibitor is omeprazole.

In another aspect, a method for treating gastro-esophageal reflux disease (GERD) and/or nocturnal acid breakthrough (NAB) is provided. The method comprises providing a first dose of a proton pump inhibitor (PPI) to deliver a first pulse of PPI; and providing a second dose of a PPI to deliver a second pulse of PPI; wherein the first pulse is released in the stomach of a patient substantially immediately after ingestion of the first dose, and the second pulse is released in the upper gastrointestinal tract of the patient substantially after ingestion of the second dose.

In one embodiment, the first and second doses are in a single dosage form.

In another embodiment, the dosage form is ingested with an evening meal.

In still another embodiment, the first and second doses are in first and second dosage forms, and wherein the second dosage form is a gastric retentive dosage form,

In another embodiment, the first and second dosage forms are ingested simultaneously or sequentially with an evening meal.

In another embodiment, a first dosage form is ingested contemporaneously with the evening meal, and the second dosage form is ingested after the evening meal but before bedtime.

In yet another embodiment, the second dosage form comprises a delivery vehicle that swells by imbibing water present in gastric fluid to a size sufficient to achieve retention in a stomach in a fed mode for release of substantially all of the second dose, and wherein the delivery vehicle comprises a component that protects at least a portion of the second dose from inactivation by exposure to acidic conditions in the stomach.

In yet another aspect, a dosage form comprising a core comprising a therapeutically effective amount of a first drug, and a shell surrounding the core is provided. The shell is comprised of a hydrophilic polymer that swells by imbibing water present in gastric fluid to a size sufficient to achieve retention in a stomach in a fed mode, and wherein the shell delays release of the first drug for a period of time substantially after ingestion, to achieve release of substantially all of the therapeutically effective amount in the stomach,

In one embodiment, the dosage form further comprises a component that protects the drug from inactivation by exposure to acidic conditions in the stomach. Exemplary protective components include an enteric coating disposed between the core and the shell or a basic excipient admixed with said drug.

In another embodiment, the period of time after ingestion for release of the dose of drug is between about 3-6 hours.

In still another aspect, a method for treating gastro-esophageal reflux disease (GERD) and/or nocturnal acid breakthrough (NAB) is provided, the method comprising providing a delayed release dosage form according to those described above, in combination with an immediate release dosage form, wherein said dosage forms comprise a proton pump inhibitor.

In another aspect, oral dosage forms suitable for the therapeutic administration of a drug such that the drug is released and absorbed in the upper GI tract at a time removed from the time of ingestion are provided. In one embodiment, the drug is acid-labile, and the dosage form comprises the drug in an enteric coating that is itself contained in a surrounding matrix that is retained in the stomach for a sustained period after ingestion. In one embodiment, the drug is a PPI.

In another aspect, oral dosage forms suitable for the therapeutic administration of a drug such that a portion of the drug in the dosage form is released in a first pulse soon after administration and the remaining portion of the drug in the dosage form is released in a second pulse at a time removed from the time of ingestion of the dosage form are provided. In one embodiment, the drug is acid-labile and subject to the first-pass effect, and the dosage form comprises two distinct portions, one in which the drug is in an enteric coating that is itself contained in a surrounding matrix that is retained in the stomach for a sustained period after ingestion, and the other in which the drug is in an enteric coating but is not contained in a matrix that is retained in the stomach. In one embodiment, the drug is a PPI.

In another aspect, a method for treating GERD and preventing NAB, the method comprising administering a PPI contemporaneously with the evening meal, such that the patient is protected from GERD due to the evening meal, and then again at bedtime, such that the patient is protected from NAB. In one embodiment of the method, the patient is administered a dosage form of a PPI, such as omeprazole, that comprises the drug in an enteric coating that is contained in a surrounding matrix that is retained in the stomach for a sustained period after ingestion to provide protection from NAB. In one embodiment, the dosage form also comprises enterically coated PPI that is not retained in the stomach, so that the dosage form provides two pulses of drug, one immediately or relatively soon after ingestion and the other that is not released until 4 to 6 to 8 or more hours after the dosage form is ingested. Thus, in one embodiment, the patient ingests once daily, contemporaneously with the evening meal, a dosage form that comprises two distinct portions, one in which the PPI is in an enteric coating that is itself contained in a surrounding matrix that is retained in the stomach for a sustained period after ingestion, and the other in which the PPI is in an enteric coating but is not contained in a matrix that is retained in the stomach. In another embodiment, the patient is administered a standard dose of a PPI, such as PRILOSEC, with the evening meal, and then is administered either another standard dose at bedtime or administered at bedtime a gastric retentive dosage form of a PPI at bedtime.

In addition to the exemplary aspects and embodiments described above, further aspects and embodiments will become apparent by reference to the drawings and by study of the following descriptions.

### BRIEF DESCRIPTION OF THE DRAWINGS

FIG. 1 is an idealized illustration of a cross-sectional view of a gastric retentive dosage form according to one embodiment;

FIG. 2 is an illustration of a cross-sectional view of a bead for use as a component in the delayed release, gastric retentive dosage forms described herein;

FIGS. 3A-3B are cross-sectional illustrations of a dosage form core comprised of a plurality of beads in a carrier matrix (FIG. 3A), and of a dosage form with a gastric retentive layer surrounding a core comprised of a plurality of beads (FIG. 3B);

FIGS. 4A-4E are illustrations of a gastric retentive delayed release dosage form comprising swellable, erodible inserts;

FIGS. 5A-5B are cross-sectional longitudinal views of dosage forms in the form of a tablet, in accord other embodiments;

FIGS. 6A-6B are model release profiles of a single pulse, delayed release dosage form (FIG. 6A) and a dosage form that provides a first immediate release pulse of drug and a second delayed release pulse of drug (FIG. 6B);

FIGS. 7A-7B are plots of the plasma concentration, in ng/mL (dashed line), and the intragastric pH (solid line) as a function of time, in hours, in subjects treated with a 20 mg dose of omeprazole at 18:00 hours in combination with a meal, and a second 20 mg dose of omeprazole at 22:00 hours;

FIG. 8 is an in vitro dissolution profile of a gastric retentive delayed release dosage form having a shell and core configuration; and

FIG. 9 is an in vitro dissolution profiles of another exemplary gastric retentive delayed release dosage form.

### DETAILED DESCRIPTION

For the convenience of the reader, the detailed description is separated into the following sections: I. Definitions; II. Dosage Forms; and III. Drugs Suitable for Administration and Methods of Use. These sections are followed by Examples of various embodiments.

### I. DEFINITIONS

“Controlled release” refers to a formulation, dosage form, or region thereof from which release of a beneficial agent is not immediate, i.e., with a “controlled release” dosage form, administration does not result in immediate release of the beneficial agent. The term is used interchangeably with “non-immediate release” as defined in Remington: The Science and Practice of Pharmacy, Nineteenth Ed. (Easton, Pa.: Mack Publishing Company, 1995). In general, the term “controlled release” includes sustained release and extended release dosage forms.

“Effective amount,” in reference to a therapeutic agent, refers to a nontoxic but sufficient amount of an agent to provide a desired beneficial effect. The amount of an agent that is “effective” may vary from individual to individual, depending on the age, weight, general condition, and other factors of the individual. An appropriate “effective” amount in any individual may be determined by one of ordinary skill in the art using routine experimentation. An “effective amount” of an agent can refer to an amount that is either therapeutically effective or prophylactically effective or both.

“Particle,” “pellet,” and “bead” are used interchangeably to refer to small, physical, sometimes spherical, units that contain a therapeutic agent. A plurality of such units are typically incorporated into a single dosage form.

“Pharmaceutically acceptable,” in reference to a component of a dosage form refers to a component that is not biologically or otherwise undesirable, i.e., the component may be incorporated into a pharmaceutical formulation and administered to a patient without causing any significant undesirable biological effects or interacting in a deleterious manner with any of the other components of the formulation in which it is contained. When the term “pharmaceutically acceptable” is used to refer to an excipient, the component has met the required standards of toxicological and manufacturing testing and/or is included on the Inactive Ingredient Guide of the U.S. Food and Drug Administration.

“Pharmacologically active” (or “active”), in reference to a “pharmacologically active” derivative or analog, refers to a derivative or analog (e.g., a salt, ester, amide, conjugate, metabolite, isomer, fragment, and the like) having the same type of pharmacological activity as the compound to which the analog or derivative is related (the “parent compound”).

“Preventing,” in reference to a disorder or unwanted physiological event in a patient, refers specifically to inhibiting or significant reducing the occurrence of symptoms associated with the disorder and/or the underlying cause of the symptoms.

“Prophylactically effective amount” refers to an amount that is effective to prevent or lessen the severity of an unwanted physiological disorder or a symptom of the disorder. Prophylactically effective amounts of a given agent will typically vary with respect to factors such as the type and severity of the disorder or disease being treated and the age, gender, weight and other factors of the patient.

“Sustained release” (synonymous with “extended release”) is used in its conventional sense to refer to a formulation, dosage form, or region thereof that provides for gradual release of a pharmacologically active agent over an extended period of time. In some embodiments, the objective of a sustained release formulation is to provide substantially constant blood levels of a pharmacologically active agent over an extended time period.

“Therapeutic agent” and “pharmacologically active agent” are used interchangeably to refer to drug compounds that are physiologically active, and to prodrugs of such compounds. Such compounds are administered for the purpose of rendering beneficial therapeutic effects and include small molecule drugs, macromolecules such as proteins, DNA and RNA.

“Therapeutically effective amount,” in reference to a therapeutic agent, refers to an amount that is effective to achieve a desired therapeutic result. Therapeutically effective amounts of a given agent will typically vary with respect to factors such as the type and severity of the disorder or disease being treated and the age, gender, weight and other factors of the patient.

“Treating”, “treat”, and “treatment” refer to reduction in severity and/or frequency of symptoms, elimination of symptoms and/or underlying cause, prevention of the occurrence of symptoms and/or their underlying cause, and improvement or remediation of damage.

As used herein, the singular forms “a,” “an” and “the” include plural referents unless the context clearly dictates otherwise. Thus, for example, “a proton pump inhibitor” refers not only to a single proton pump inhibitor but also to a combination of two or more different proton pump inhibitors, and “an excipient” refers both to a combination of excipients as well as to a single excipient.

As used herein, the phrases “for example,” “for instance,” “such as,” and “including” are meant to introduce examples to illustrate more general subject matter. These examples are provided only as an aid for understanding the disclosure, and are not meant to be limiting in any fashion.

Unless defined otherwise, all technical and scientific terms used herein have the meaning commonly understood by one of ordinary skill in the art to which the subject matter herein pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the present disclosure or its claims.

### II. EXEMPLARY DELAYED RELEASE, GASTRIC RETENTIVE DOSAGE FORMS

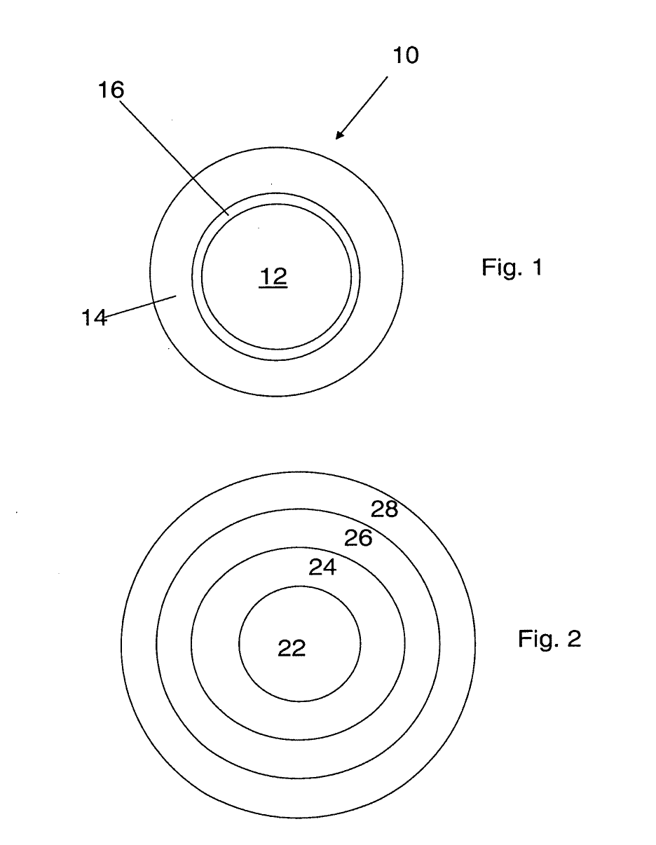

Dosage forms described herein are intended for oral administration, and are suitable for administration of a variety of therapeutic drugs. The dosage forms are particularly suited for administration of drugs that are preferentially absorbed in the upper GI tract, and/or for administration of drugs that are inactivated or degraded by conditions in the upper GI tract. The dosage forms are also particularly suited for administration of drugs that are subject to the first-pass effect. Various embodiments of the dosage form are described with reference to FIGS. 1-4, now to be described.

In a first embodiment, the dosage form is designed to release a dose of drug to the stomach at a time substantially after ingestion of the dosage form. An exemplary gastric retentive dosage form that provides delayed release of its active agent is shown in FIG. 1. Dosage form 10 is comprised of a drug core that is surrounded or encased by a polymeric shell 14. An optional protective layer 16 can be disposed between the drug core and the shell, and is typically included in the dosage form when the drug is degraded or inactivated by the stomach conditions, for example, acid-labile drugs. Shell 14 is comprised of a polymer that swells unrestrained dimensionally in water, such as in the water present in gastric fluid. Swelling of shell 14 increases the size of the dosage form to a size sufficient for retention in the stomach in the fed mode, i.e., to a size equal to or greater than the size of the opening of the pyloric sphincter in the fed mode. The mean pyloric diameter in the fed mode is between 0.9-1.4 cm, with an average of about 1.2 cm.

Drug in core 12 is released from dosage form 10 upon, for example, erosion of shell 14 or upon a combination of erosion of shell 14 and diffusion of drug across shell 14. In a preferred embodiment, shell 14 erodes after ingestion of the dosage form to achieve release of the drug in core 12 in a single “pulse” or bolus dose, as opposed on a sustained or extended release type of delivery. The properties of shell 14, e.g., the polymer from which it is fabricated, the presence of any additives or excipients, and its thickness, determine the rate of erosion and swelling, and a skilled artisan can appreciate the approaches to varying these parameters. Shell 14 is preferably a hydrophilic, erodible polymer, and exemplary polymers are described below.

Core 12 in dosage form 10 comprises the active agent or drug and any other desired excipients. These are mixed together typically as solid powders or granules and compressed to form the active core. The core is typically substantially homogeneous, such that the active agent is distributed evenly throughout the core. Suitable excipients include, for example, inert carriers and the like.

The gastric retentive dosage forms of this embodiment typically have a diameter prior to swelling that is within the range of about 5 mm to about 20 mm, more typically within the range of about 5 mm to about 15 mm or of about 5 mm to about 12 mm or of about 7 mm to 12 mm. Mini-tablets can also be prepared having diameters within the range of about 1 mm to about 8 mm, or about 1 mm to about 5 mm, or about 2 mm to about 5 mm. Once administered to the GI tract, the dosage form contacts gastric juices and swells to a diameter that provides for gastric retention, typically at least 1.5 to 2 times the size of the dosage form prior to administration. In some embodiments, the swelled form of the dosage form is in the range of about 10 mm to about 25 mm or about 10 mm to about 20 mm.

In addition to achieving an increase in size of the dosage form, swelling of the outer polymeric shell in the dosage form results in a delay in delivery or release of drug from the dosage form such that the dose of drug is released in the stomach at a time substantially after ingestion of the dosage form. By “substantially after ingestion” it is intended that the dose of drug contained in the dosage form in released between about 2-6 hours, more preferably 3-5 hours, still more preferably 3-4 hours, and still more preferably 2-5 hours or 2-4 hours after oral ingestion. In addition, the dose of drug is released as a burst or pulse of drug, as opposed to a sustained or extended release.

As mentioned above, core 12 in dosage form 10 can be comprised of drug in solid form compressed with one or more excipients to form the core, e.g., a conventional tablet of compressed solid drug. In another embodiment, core 12 is comprised of a plurality of particles or beads that are compressed to form a core, and an idealized exemplary particle or bead is illustrated in FIG. 2.

As seen in FIG. 2, bead 20 is comprised of a bead core 22, a drug coating 24 surrounding the bead core, an optional sub-coat layer 26, and an optional protective coating 28. The bead core serves as a supporting substrate, and is preferably comprised of an inert, pharmaceutically-acceptable material, such as a starch, a sugar, microcrystalline cellulose, and the like. Examples of suitable materials include nonpareils; SUGLETS® (supplied by NP Pharm, France, and composed of not more than 92% sucrose and (the remainder) maize starch); and CELPHERE® (supplied by Asahi Kasei, Japan, and composed of microcrystalline cellulose). The size of the bead core may be, for example, about 300-1200 μm, and is preferably between about 355-425 μm, about 600-710 μm, and about 1000-1180 μm.

Drug layer 24 comprises the active agent or drug and, optionally, any desired pharmaceutically acceptable excipients. Typical pharmaceutically acceptable excipients include, for example, carriers such as hydroxypropyl methylcellulose (HPMC, commonly called hypromellose), surfactants such as TWEEN® 80 (polyethylene glycol sorbitan monooleate), and other excipients described herein and/or known in the art. The thickness of the layer is typically determined by the manufacturing process percentage weight gain specification but can be, for example, within the range of about 100-250 μm, and may vary with bead core size. The typical mass of this layer is 10 to 50% of the bead core mass, depending on the size of the bead core.

Optional sub-coat layer 26 is typically employed when it is desirable to protect the drug in the drug layer from a component in the protective layer. For example, a protective layer that serves as an enteric coating may comprise an acidic component, and the optional sub-coat would be included to protect the drug from such an acidic component. The sub-coat layer should allow for relatively immediate release of the drug layer once the protective layer is removed. Examples of suitable materials for the sub-coat layer include OPADRY® YS-1-19025-A-Clear and OPADRY-03K (supplied by Colorcon, Pennsylvania). The sub-coat layer may also contain additional excipients, including any described elsewhere herein, as well as alkaline compounds such as bases, salts, and the like. The thickness of the sub-coat layer is typically determined by the manufacturing process percentage weight gain specification but can be, for example, within the range of about 10-50 μm. The typical mass of this layer is 3 to 5% of the mass of the bead core.

Protective coating 28 is an optional layer, and is included, for example, when the drug is acid-labile and protecting or stabilizing the drug from the environment of use is desired. The protective coating, when included, is, in a preferred embodiment, is an enteric coating layer that protects the drug layer from degradation by gastric acid. An example of a material for use in forming the enteric coating layer is ACRYL-EZE® (methacrylic acid copolymer, supplied by Colorcon, Pennsylvania). The plastic properties of this coat can be optimized by adding a plasticizer, including but not limited to plasticizers such as triethyl citrate (TEC) with or without a mixture of EUDRAGIT L30 D-55 (for acid protection) and EUDRAGIT NE 30 (a plasticizer) (EUDRAGIT is marketed by Degussa). The enteric coating layer may have additional excipients such as anti-adherent agents (e.g., talc) or anti-foaming agents (e.g., a simethicone emulsion). The thickness of the layer is typically determined by the manufacturing process percentage weight gain specification but can be, for example, within the range of about 100-250 μm, and may vary with bead core size. The typical mass of this layer is typically a minimum of 30% of the mass of the bead core. The typical mass of EUDRAGIT polymers per unit area of surface to be coated is 4 to 6 mg/cm².

It is also contemplated that the protective coating can be a coating that erodes at a controlled rate, such that the drug is released as a burst or pulse at a time defined by the rate of erosion. For example, the protective layer can be a polymer that erodes, and the thickness of the protective layer is selected such that the layer is eroded within a defined time after ingestion to achieve release of the drug.

It is also contemplated that the protective coating can be a stabilizing component that is added to the dosage form, such as a basic compound.

Each of layers 24, 28, and optional layer 26, may be applied to the bead core in the form of a solution, suspension, or emulsion, and preferably an aqueous solution. Typically, in the final dosage form, all or most of the water and/or any organic solvent used in the manufacturing process has been removed from each layer.

In another embodiment, drug pellets are manufactured, rather than a bead as described above. A drug pellet is prepared, for example, by mixing the drug with a binder (i.e., microcrystalline cellulose), extruding and spheronizing the mixture to create pellets containing drug, preferably at a weight percentage of 1 to 99%, such as between 20 and 80% drug. The extrudate can be coated with a protective coating, such as an enteric coating, and with an optional subcoat disposed between the drug pellet and the protective coating.

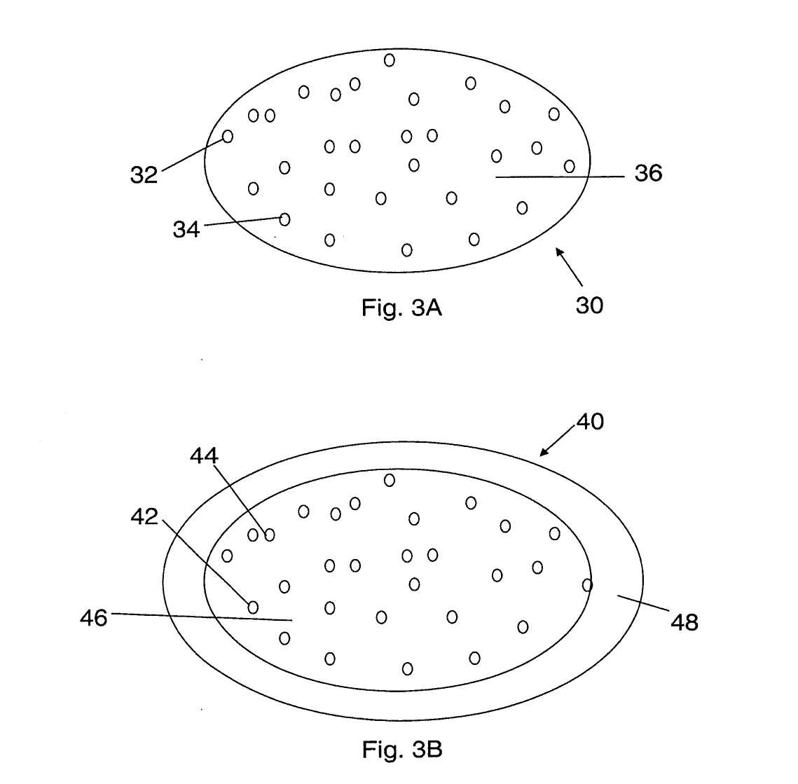

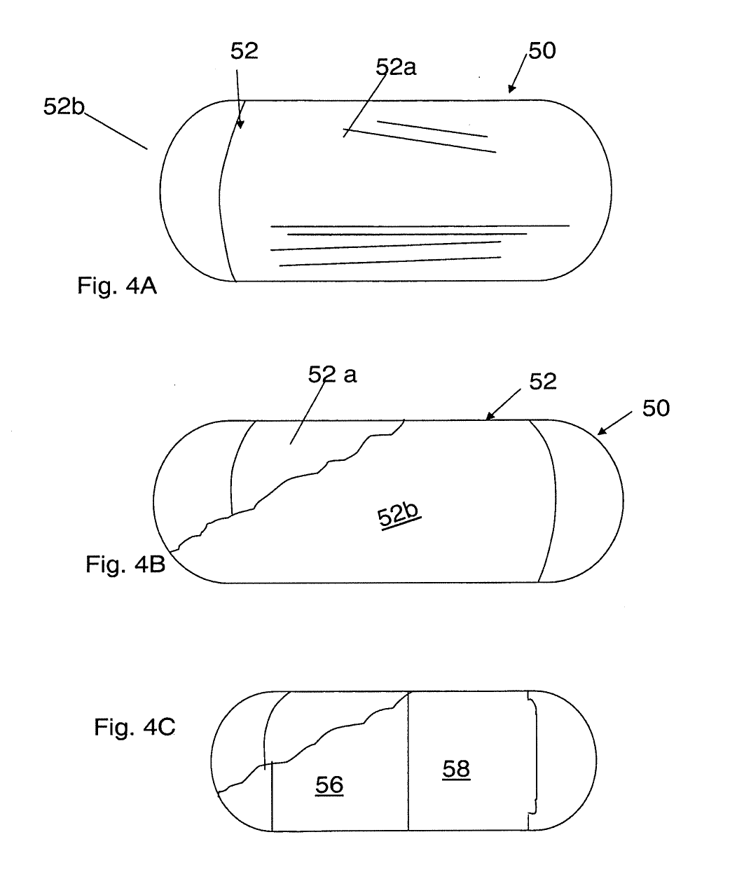

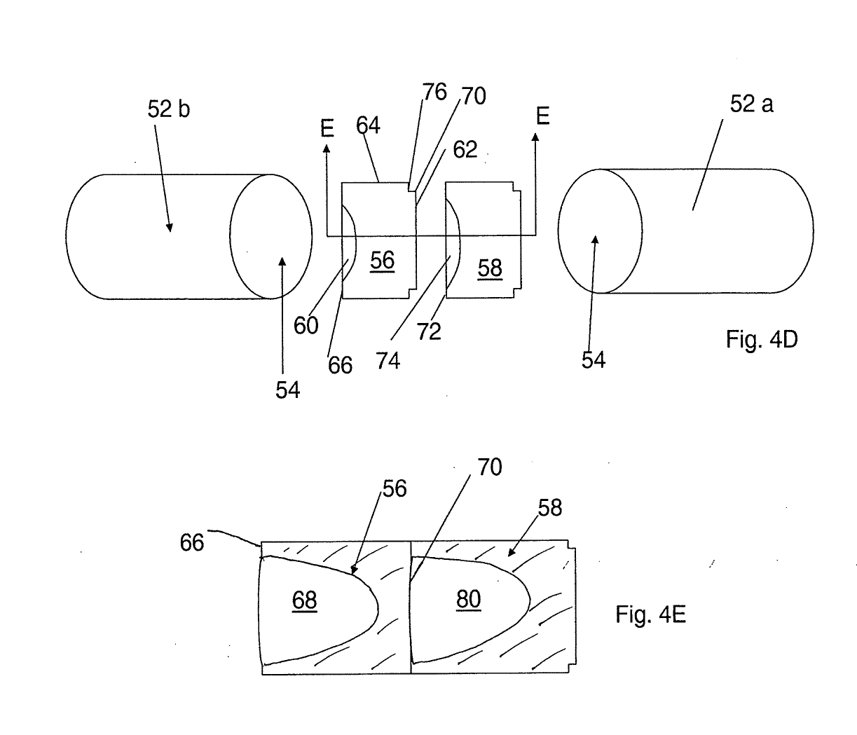

Once formed, the beads or drug pellets can be compressed alone or with appropriate excipients into a core for use in a dosage form, such as that depicted in FIG. 1. The pellets or beads can also be used to fabricate other dosage forms, and these embodiments are now described with reference to FIGS. 3-4.

FIG. 3A illustrates a gastric-retentive dosage form 30 comprised of a plurality of beads, such as beads 32, 34, dispersed in a matrix 36. In one embodiment, matrix 36 is a polymeric matrix comprised of a hydrophilic polymer that swells in water, such that the dosage form swells unrestrained dimensionally upon imbibing water in gastric fluid to a size the inhibits its passage through the pyloric sphincter in the fed mode. Such a dosage form provides gastric retention, to achieve release of the drug in the plurality of beads in the stomach, and delayed release. The delayed release is achieved by appropriate selection of the polymeric matrix and the rate and extent of its erosion after ingestion. The rate and extent of its erosion determine the rate at which fluid reaches the protective coating of each bead dispersed the polymer matrix, solubilization of the protective coating, and eventual release of the drug in the drug layer of each bead.

FIG. 3B illustrates another exemplary gastric-retentive dosage form 40 that incorporates a plurality of pellets or beads, such as the beads depicted in FIG. 2. In this embodiment, beads, such as beads 42, 44, are dispersed in a matrix 46. Matrix 46 in this embodiment is comprised of the beads compressed with one or more excipients. Matrix 46 is surrounded by a polymer coating 48 that is comprised of a swellable, erodible hydrophilic polymer. The hydrophilic polymer swells in water, such that the dosage form swells unrestrained dimensionally upon imbibing water in gastric fluid to a size that inhibits its passage through the stomach's pyloric sphincter in the fed mode. Such a dosage form provides gastric retention, to achieve release of the drug in the plurality of beads in the stomach, and delayed release. The delayed release is achieved by appropriate selection of the polymer in the polymer coating and the rate and extent of its erosion after ingestion. The rate and extent of its erosion determine the rate at which fluid reaches matrix 46, to solubilize the protective coating on each bead in the matrix, and provide release of the drug in the drug layer of each bead. It will be appreciated that gastric retentive properties can also be achieved by coating each bead with a gastric retentive coating layer, such that each active bead independently has gastric retentive characteristics.

Water-swellable, erodible polymers suitable for use herein are those that swell in a dimensionally unrestrained manner upon contact with water, and gradually erode over time. Examples of such polymers include cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; polyalkylene oxides, such as polyethylene glycols, particularly high molecular weight polyethylene glycols; chitosan; poly(vinyl alcohol); xanthan gum; maleic anhydride copolymers; poly(vinyl pyrrolidone); starch and starch-based polymers; maltodextrins; poly (2-ethyl-2-oxazoline); poly(ethyleneimine); polyurethane; hydrogels; crosslinked polyacrylic acids; and combinations or blends of any of the foregoing.

Further examples are copolymers, including block copolymers and graft polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

Preferred swellable, erodible hydrophilic polymers suitable for forming the gastric retentive portion of the dosage forms described herein are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 9×10⁵ Daltons to about 8×10⁶ Daltons. A preferred molecular weight poly(ethylene oxide) polymer is about 5×10⁶ Daltons and is commercially available from The Dow Chemical Company (Midland, Mich.) referred to as SENTRY® POLYOX® water-soluble resins, NF (National Formulary) grade WSR Coagulant. The viscosity of a 1% water solution of the polymer at 25° C. preferably ranges from 4500 to 7500 centipoise.

Yet another embodiment of a dosage form that provides for delayed, gastric-retentive release of a drug is illustrated in FIGS. 4A-4E. Dosage form 50 is comprised of a capsule 52 having a first portion 52a and a second portion 52b, seen best in the exploded view of FIG. 4D and the view of FIG. 4B where a part of outer layer 52a is removed. First and second portions 52a, 52b, are sized such that the second portion is removably insertable into the first portion, to form capsule 52 that has an interior cavity 54.

Contained within the interior cavity of the capsule is one, two, three, or more inserts, such as inserts 56, 58 visible in FIGS. 4C-4D. Each insert is comprised of an erodible, swellable, hydrophilic polymer, and is shaped for congruency or nesting arrangement with an adjacent insert. In the embodiment shown, insert 56 has a first end 60 and a second end 62 and a wall 64. First end 60 has a rim 66 of a thickness l that defines an internal diameter of a cavity 68, visible in the cross-sectional view of insert 56 shown in FIG. 4E. End 62 of insert 56 has a protruding lip 70 that is sized for sealing engagement or insertion into an adjacent insert, such as insert 58. As best seen in FIG. 4E, lip 70 inserts into an end of insert 58, and rim 72 on end 74 of insert 58 mates with beveled edge 76 of end 62 on insert 56. As will be discussed below, the engagement of adjacent inserts, and specifically engagement of a rim of a first insert with an edge of a second insert, creates a seal that closes the cavity within an insert from the environment of use, delaying release of the cavity's contents for a period of time. In the embodiment of FIG. 4E, contents in cavity 80 of insert 58 is sealed by engagement with adjacent insert 56, to delay release of content within cavity 80.

The gastric retentive and delayed release properties of the dosage form of FIGS. 4A-4E are best understood by describing events after oral administration. A dosage form as depicted in FIGS. 4A-4E is prepared to include a first insert and a second insert. The cavity of each insert is filled with drug, in the form of drug pellets or, in a preferred embodiment, in the form of beads as shown in FIG. 2. The drug-loaded inserts are inserted into a capsule, such as a pressure fitting gelatin capsule that dissolves, erodes, or otherwise disintegrates upon contact with gastric juices. The dosage form is ingested orally, and upon contact with gastric fluid in the stomach the capsule dissolves, exposing the inserts to the stomach environment. The term “ingested” intends that the dosage form is taken into the body by the mouth. As discussed above with reference to FIG. 4E, the first and second inserts are in a nested arrangement, such that upon dissolution of the outer capsule, the cavity of a first insert is exposed to the environment and the cavity of the second insert remains sealed by an end of the adjacent, nested insert. The first drug dose contained in cavity of the first insert is released into the stomach as a first pulse or bolus dose. This first drug dose is essentially an immediate release dose, since the dissolution of the capsule is rapid upon ingestion. Thus, the first dose of drug is delivered to the patient substantially immediately after oral administration. By “substantially immediately” is intended less than 60 minutes, preferably less than 30 minutes, and more preferably less than 20 minutes, and still more preferably between 10-30 minutes after ingestion of the dosage form.

Once the capsule shell dissolves or otherwise disintegrates, the erodible inserts are exposed to the surrounding liquid (e.g., gastric juices in the stomach of a patient). Water imbibation causes the erodible inserts to fuse together via polymeric entanglement following exposure to gastric fluids or other aqueous environment and swell to a size that is retained in the stomach for a period of time. That is, the inserts form in situ a seal that closes one of the cavities and prevents release of its contents for a period of time. During this period, the gastric retentive erodible inserts begin to erode and, after a given period of time, erosion of the erodible inserts allows any material contained within the cavity to empty from the dosage form into the surrounding environment (e.g., the stomach). The period of time required to breach the seal will depend on a variety of factors such as the thickness of the walls of the erodible inserts, the material from which the erodible inserts are made, the pH of the liquid eroding the insert, the amount of mechanical turbulence in the environment, and other factors. Selection of the materials and optimization of the wall thickness to obtain the desired release time in view of such factors and variables is within the capabilities of the skilled artisan upon consideration of this disclosure and references cited herein.

In particular, and with reference to FIG. 4E, the dimensions of the inserts and the polymer from which the inserts are manufactured influence the time for the eventual release of the second dose of drug contained in the second insert. In particular, the thickness of the rim surrounding the cavity opening, such as rim 72 in insert 58 of FIG. 4E, and the dimensions of the beveled edge, as well as dimensions of the insert cavity and the insert's overall size, influence the time required for erosion of the insert to an extent sufficient to achieve release of the contents in the second insert cavity. Because the inserts swell to a size that achieves retention in the stomach, the release of the second cavity's contents occurs in the stomach, resulting in two pulses of drug delivered to the stomach.

The dosage forms described above are gastric retentive due largely to a layer or component fabricated from a hydrophilic, swellable polymer. The gastric retentive component is also referred to herein as a delivery vehicle, and such a delivery vehicle is specifically exemplified by the polymeric shell of the dosage form in FIG. 1 and FIG. 3B, and by the polymeric matrix of FIG. 3A, and by the inserts of FIGS. 4A-4E.

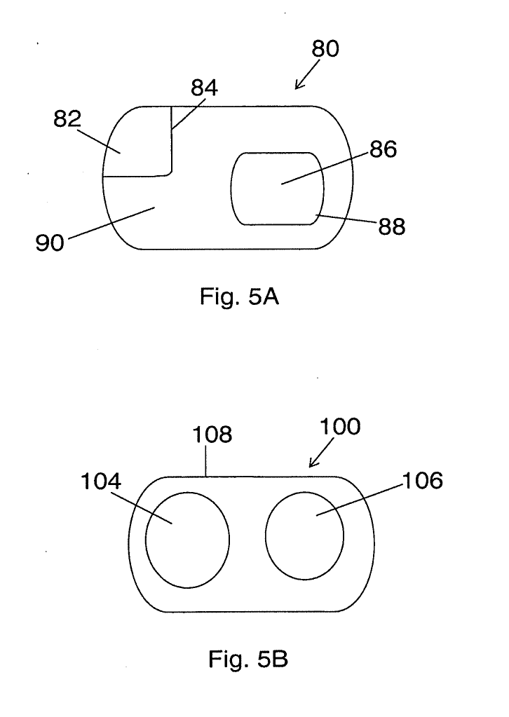

It will be appreciated that the pulsatile, delayed release dosage forms described above are merely exemplary, and that a wide variety of dosage form configurations are contemplated, and can be readily designed by a skilled artisan. Further exemplary dosage form configurations are illustrated in FIG. 5. FIG. 5 shows a cross-sectional longitudinal view of a dosage form 80 in the form of a tablet. The tablet is comprised of a first drug dose 82 confined to a first region 84, and a second drug dose 86 confined to a second region 88. A tablet matrix 90 separates the first and second regions, 84, 88, and is comprised of a swellable, erodible hydrophilic polymer.

First drug dose 82 is positioned for exposure to external surface 92 of the dosage form. Second drug dose 86 is positioned so that it is surrounded or encased by the tablet matrix. As can be appreciated, this positioning of the drug doses achieves the desired pulsed release profile. Upon ingestion of dosage form 80, first drug dose 82 is released essentially immediately to the stomach, as the first drug dose is exposed to the tablet surface and accessible for solubilization and release. Tablet matrix 90 swells upon contact with water in gastric fluid, inhibiting release of drug from the second drug region that is entirely surrounded by the now swollen polymer matrix. The tablet matrix swells to an extent sufficient to prevent passage of the dosage form through the pyloric sphincter when in the fed mode. Release of drug from the second region is delayed for a period of time determined in part by the polymer and other materials in the matrix, the thickness of the matrix, the stomach conditions, and other factors. The second dose of drug is released in the stomach upon erosion of the tablet matrix to a degree sufficient to expose the second drug dose to the stomach environment. Release of the second dose occurs as a burst or pulse.

The first and second drug doses can be comprised of solid drug and any desired excipients, such as a base or other pH stabilizing agent for acid-labile drugs. Either or both of the first and second drug doses can also be comprised of the beads described above, wherein a plurality of beads sufficient to provide the desired dose are compressed, alone or with any desired excipients, into the first and second regions during the tableting manufacturing process. It will also be appreciated that all or a portion of the dosage form can be coated with an external enteric coating or an additional drug coating.

FIG. 5B illustrates another embodiment of a dosage form tablet, that provides a dual delayed pulsed release, and an optional immediate release pulse of drug. Dosage form 100 is comprised of a tablet matrix 102 having first region 104 and second region 106 containing first and second drug doses. The drug dose in each of the first and second regions is released from the dosage form at a time determined at least in part by the tablet matrix and the size the position of each region in the tablet. Adjusting the size and location of each region, as well as the selection of the polymer forming the matrix and the thickness of the regions surrounding each of the first and second regions influences the time required for erosion of the matrix and release of the drug dose in the first and second regions. The external surface 108 of the dosage form can optionally include a drug coating that provides an immediate release of drug upon ingestion.

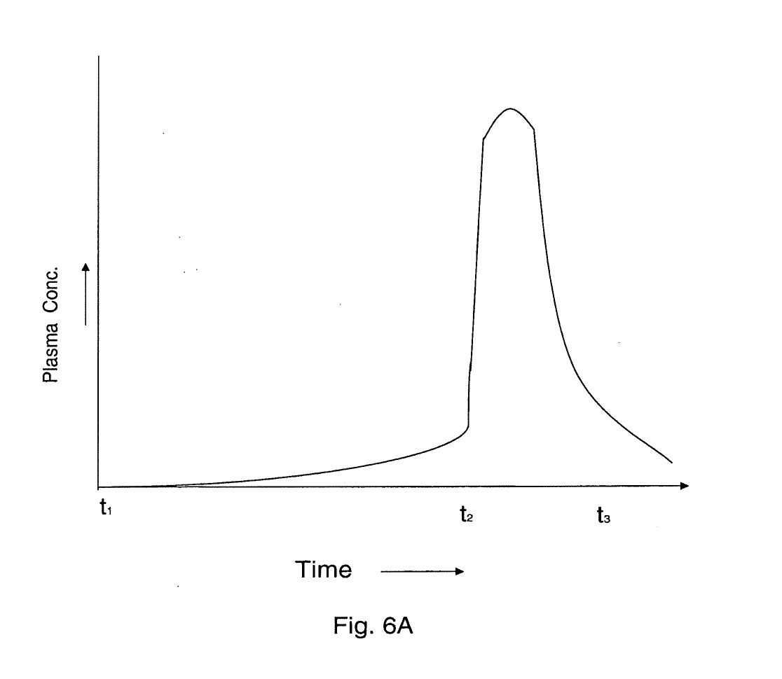

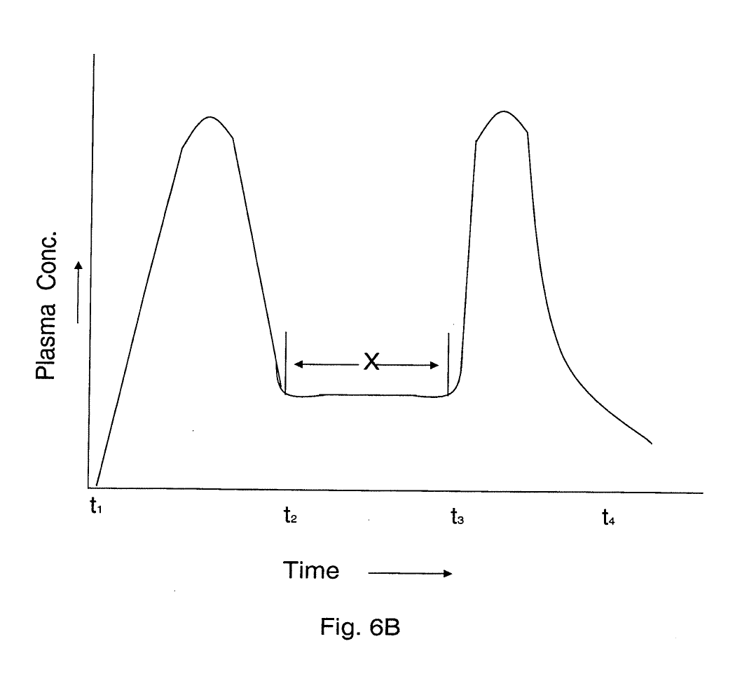

FIGS. 6A-6B illustrate release of drug from dosage forms described above. FIG. 6A shows a single pulse, delayed release delivery profile, where a bolus of drug is delivered at time t₂, which is a time substantially removed from ingestion of the dosage form at time t₃. Time t₂ is preferably 2, 3, 4, 5, or 6 hours, or between 2-3 hours, 2-4 hours, or 2-5 hours after ingestion of the dosage form. The dosage forms illustrated in FIG. 1 and in FIGS. 3A-3B each provide a single pulse, delayed release delivery of drug to the stomach. In addition, the dosage form depicted in FIGS. 4A-4E can also provide a single, delayed pulse release of drug by leaving the cavity in the first insert empty and providing a first dose of drug in the second insert that is sealed in situ upon swelling of the inserts.

FIG. 6B illustrates release a pulsed delivery profile, where a first pulse of drug is delivered at time t₁ and a second pulse of drug is released from the dosage form at time t₃. Time t₁ is substantially immediately after ingestion of the dosage form, e.g., within 10-30 minutes after ingestion. Time t₃ is a time removed from ingestion of the dosage form, and is preferably 2, 3, 4, 5, or 6 hours, or between 2-3 hours, 2-4 hours, or 2-5 hours after ingestion of the dosage form. The gastric retentive nature of the dosage forms ensures that the second pulse of drug is administered in the stomach and upper GI tract, thus providing a first pulse and a second delayed pulse delivered in the stomach of patient. It will be appreciated that the dosage forms of FIG. 1 and FIGS. 3A-3B can be manufactured to include an immediate release drug layer on the external surface of the dosage form, the immediate release drug layer providing the first pulsed dose of drug. In this way, each of the dosage forms described above can be prepared to provide a first and second pulsed drug release.

As noted above, in some embodiments, the dosage forms include a plurality of beads, wherein the plurality comprise a desired dose of drug. The first dose of drug that is immediately released is associated with a first plurality of beads, and the second or subsequent dose(s) of drug are associated with second and subsequent plurality of beads. It is contemplated that the size of the beads in the one or more pluralities of beads can be the same or different. For example, to achieve a bolus release of drug from a first plurality of beads in a narrow window of time, i.e., a short time between t₃ and t₄ in FIG. 6B, a collection of beads having an outer diameter in the range of about 2 mm or less, preferably 1 mm or less, is preferred. The lower outer diameter size limit is determined by manufacturing constraints, and the available sizes of bead core materials. A typical minimum size is on the order of 0.1 mm, or 0.2 mm, or 0.5 mm. Beads of a smaller size will provide a release of drug dose in a narrow window of time. The beads contained in the delayed drug pulse can be larger than 2 mm, and are preferably contained in a polymer matrix that swells to a minimum outer diameter size of 4 mm or more, and preferably of between about 4 mm to about 8 mm, on that the size of the bead collection exceeds the mean pyloric diameter in the fed mode of about 1.2 cm, to promote retention of the collection of beads in the fed mode.

With reference again to the dosage form in FIGS. 4A-4E, it will be appreciated that each erodible insert in a dosage form may be identical in shape, or may differ in shape (e.g., a “top” and a “bottom” insert) from other erodible insert(s) in the dosage. In one embodiment, the erodible inserts have a shape having a male end and a female end, and in another embodiment, each erodible insert comprises both a male component on one side and a female component on the opposite side. The male and female connecting portions may be tapered, stepped, screw-like (e.g., helical), or a combination thereof. In one embodiment, joining the male end or side of one erodible insert to the female end or side of another erodible insert creates a void large enough to contain the desired amount of drug to be released in a delayed pulse.

The delayed pulse drug reservoir can comprise enteric coated drug-containing beads, such as those described previously, as well as any desired excipients as appropriate. Alternatively, the delayed pulse drug reservoir may comprise a mini-tablet comprising a drug-containing core and, if appropriate, an enteric coating layer. Such a mini-tablet is similar to the dosage form in FIG. 1 above, although the gastric retention provided by the erodible inserts renders the requirement for an outer swellable polymeric coating around the drug core unnecessary, and a mini-tablet may be prepared without a gastric retentive coating layer when the mini-tablet(s) is/are placed in the cavity of an insert.

In a preferred embodiment, the delayed pulse dosage forms described above comprise a proton pump inhibitor compound, such as omeprazole. Omeprazole particles incorporated into a core that has an enteric coating and a gastric retentive coating, such as the dosage from illustrated in FIGS. 1 and 3B, are contemplated. The acid protected gastric retentive tablet core can optionally be further coated with immediate release particles or an immediate release coating layer. Alternatively, each of the omeprazole particles can have an enteric coating and a gastric retentive coating, and such particles can be pressed into a tablet or filled into a capsule along with a matrix comprising the initial pulse of omeprazole and any suitable excipients. Again, the initial pulse may be present in the form of immediate release particles or a more homogeneous mixture of omeprazole with excipients (and, optionally, abuse).

In a preferred embodiment, a dosage form as depicted in FIGS. 4A-4E is prepared with a first dose of omeprazole for immediate release contained with a first cavity of an insert and/or within void spaces between the inserts and the capsule. The immediate release omeprazole is formulated with a protective component, such as a basic material or in the form of drug pellets or beads with an enteric coating (as exemplified in FIG. 2). Alternatively, the immediate release pulse may be present as a coating on the erodible inserts. A delayed pulse release of a second dose of omeprazole is contained within a second cavity of a second insert, for release at a time well after ingestion of the dosage form.

In yet another alternative embodiment, a bilayer tablet is prepared comprising an immediate release layer and a delayed release layer. Bilayer tablets are known in the art, and the skilled artisan will be capable of their preparation using the methods disclosed herein along with commonly available methods. Other alternatives for incorporating the immediate release pulse with the delayed release pulse will be apparent to those of skill in the art upon consideration of this disclosure.

Dosage forms that provide more than two pulses of drug release are contemplated, and a skilled artisan will appreciate the modifications to the dosage forms described above to provide a third, fourth or further drug dose pulse. Multiple pulses are possible using variations of the embodiments described herein. For dosage forms using erodible inserts, a plurality of pulses may be obtained by using more than two identical or different erodible inserts in the dosage form, in which the different inserts provide different erosion times. For dosage forms comprising tablet cores and/or beads, additional pulses may be obtained by using a plurality of gastric retentive layers alternated with layers comprising the active agent.

For any of the embodiments, the optional initial (i.e., immediate release) pulse of drug can be combined with the delayed release pulse in any suitable manner. In general, the initial pulse of drug is released in the stomach rapidly upon administration. The second (i.e., delayed) pulse of active agent may be prepared such that it follows administration of the dosage form at any time, and the skilled artisan will understand in view of the disclosure herein how to provide the desired time of release. For example, increasing the thickness of the walls of the gastric retentive insert will increase the time delay between administration of the dosage form and release of the delayed pulse of drug. The optimal time delay between administration of the dosage form and release of the delayed pulse will depend on a number of factors, such as the condition being treated, the physical characteristics and daily routine of the patient being treated, and the like.

In various embodiments, the delayed pulse will release active agent to the duodenum and small intestines of the patient within about 2 to 12 hours after administration of the dosage form, for example within about 3 to 9 hours, or for example within about 4-6 hours. Release of the delayed release pulse may be targeted for about 3, 4, 5, 6, 7, 8, 9, 10, 11, or 12 hours after administration of the dosage form. As a further example, release of the delayed release pulse may be target for between about 2 to 4 hours, or between about 3 to 5 hours, or between about 5 to 7 hours, or between about 6 to 8 hours after administration of the dosage form.

Generally, the initial pulse (when present) releases a dose of active agent or drug that is between about 0.25 and 20 times the dose of active agent or drug that is present in the delayed pulse. Measured as a ratio, the drug dose ratio of the initial to delayed pulses may be about 0.25 to 4, or 0.5 to 2, or 0.75 to 1.25, and can be 1 to 1. The amount of active agent in the formulation typically ranges from about 0.05 wt. % to about 95 wt % based on the total weight of the formulation. For example, the amount of active agent may range from about 0.05 wt % to about 50 wt %, or from about 0.1 wt % to about 25 wt %, or from about 1 wt % to about 15 wt %. Alternatively, the amount of active agent in the formulation may be measured so as to achieve a desired dose, concentration, plasma level upon administration, or the like. The amount of active agent may be calculated to achieve a specific dose (i.e., unit weight of active agent per unit weight of patient) of active agent. Furthermore, the treatment regimen may be designed to sustain a predetermined systemic level of active agent. For example, formulations and treatment regimen may be designed to provide an amount of active agent that ranges from about 0.001 mg/kg/day to about 100 mg/kg/day for an adult. As a further example, the amount of active agent may range from about 0.1 mg/kg/day to about 50 mg/kg/day, about 0.1 mg/kg/day to about 25 mg/kg/day, or about 1 mg/kg/day to about 10 mg/kg/day. One of skill in the art will appreciate that dosages may vary depending on a variety of factors, including physical characteristics of the patient and duration of treatment regimen.

Numerous materials useful for manufacturing dosage forms described herein are described in Remington The Science and Practice of Pharmacy, 20^{th} edition (Lippincott Williams & Wilkins, 2000) and Ansel et al., Pharmaceutical Dosage Forms and Drug Delivery Systems, 6^{th} Ed. (Media, P A: Williams & Wilkins, 1995). Pharmaceutically acceptable additives or excipients include binders (e.g., ethyl cellulose, gelatin, gums, polyethylene glycol, polyvinylpyrrolidone, polyvinylalcohol, starch, sugars, waxes), disintegrants, coloring agents, diluents (e.g., calcium sulfate, cellulose, dicalcium phosphate, kaolin, lactose, mannitol, microcrystalline cellulose, sodium chloride, sorbitol, starch, sucrose), flavoring agents, glidants (e.g., colloidal silicon dioxide, talc), and lubricants (e.g., calcium stearate, glyceryl behenate, hydrogenated vegetable oils, magnesium stearate, polyethylene glycol, sodium stearyl fumarate, stearic acid, stearyl behenate, talc), sweeteners, polymers, waxes, and solubility-retarding materials. The dosage forms described herein can be made by techniques that are well established in the art, including wet granulation, fluid-bed granulation, dry granulation, direct compression, and so forth.

In one embodiment, the drug is acid-labile, and the dosage form comprises the drug in an enteric coating that is itself contained in a surrounding matrix that is retained in the stomach for a sustained period after ingestion. Oral dosage forms suitable for the therapeutic administration of a drug are provided, such that a portion of the drug in the dosage form is released in a first pulse soon after administration and the remaining portion of the drug in the dosage form is released in a second pulse at a time removed from the time of ingestion of the dosage form. Thus, these two different dosage forms differ in that the second delivers two different “pulses” of drug release, the first coming relatively soon after ingestion (the “initial pulse”) and the second (the “delayed pulse”) much later in time.

In a first example, in which one presumes that the drug to be administered is acid-labile or is targeted for release in the stomach and/or small intestine, the initial pulse results from a layer of acid-protected immediate release particles incorporated into the dosage form. The acid-protected immediate release particles can be, for example, particles comprising the drug of interest and a pharmaceutically acceptable carrier in an immediate release core, wherein the immediate release core is coated with an enteric coating to protect it from the acidic conditions of the stomach. Alternatively or in addition, a base may be incorporated into the immediate release core to provide protection from acidic conditions. The enteric coated particles are incorporated into the dosage form such that they are released rapidly after administration. For example, the particles (along with other pharmaceutically acceptable excipients such as an erodible polymer) can be incorporated into the outermost layer of the dosage form. Upon administration of the dosage form, the outermost layer rapidly dissolves, erodes, or otherwise degrades and so releases the particles of the first pulse into the upper GI tract. In a second example, the initial pulse results from an immediate release coating layer, which consists of a non-particulate mixture of the active agent or drug, an optional base, and an optional pharmaceutically acceptable carrier such as an erodible polymer. Upon administration, the immediate-release drug layer erodes or dissolves in the stomach, thereby releasing the first pulse of active agent.

The delayed pulse of drug released from the dosage forms is provided by incorporating the drug into a gastric-retentive matrix. If the drug to be administered is acid sensitive, then, as for the drug delivered in the initial pulse, the drug delivered in the delayed pulse is acid protected by using, for example, an enteric coating and/or is formulated with abuse.

The dosage forms are intended for oral dosage administration. Preferred oral dosage forms include tablets, capsules, and the like. Tablets may comprise, for example, a flavored base such as compressed lactose, sucrose and acacia or tragacanth and an effective amount of an active agent. Tablets can be prepared by common tabletting methods that involve mixing, comminution, and fabrication steps commonly practiced by and well known to those skilled in the art of manufacturing drug formulations. Examples of such techniques are: (1) direct compression using appropriate punches and dies, typically fitted to a suitable rotary tabletting press; (2) injection or compression molding; (3) granulation by fluid bed, by low or high shear granulation, or by roller compaction, followed by compression; (4) extrusion of a paste into a mold or to an extrudate to be cut into lengths; (5) coating techniques, including pan-coating, fluid-bed coating and bottom spray methods (Wurster) and other film coating methods; and (6) powder layering.

When tablets are made by direct compression, the addition of lubricants may be helpful and is sometimes important to promote powder flow and to prevent breaking of the tablet when the pressure is relieved. Examples of typical lubricants are magnesium stearate (in a concentration of from 0.25% to 3% by weight, preferably about 1% or less by weight, in the powder mix), stearic acid (0.5% to 3% by weight), and hydrogenated vegetable oil (preferably hydrogenated and refined triglycerides of stearic and palmitic acids at about 1% to 5% by weight, most preferably about 2% by weight). Additional excipients may be added as granulating aids (low molecular weight HPMC at 2-5% by weight, for example), binders (microcrystalline cellulose, for example), and additives to enhance powder flowability, tablet hardness, and tablet friability and to reduce adherence to the die wall. Other fillers and binders include, but are not limited to, lactose (anhydrous or monohydrate), maltodextrins, sugars, starches, and other common pharmaceutical excipients. These additional excipients may constitute from 1% to 50% by weight, and in some cases more, of the tablet.

In addition to the foregoing components, it may be necessary or desirable in some cases (depending, for instance, on the particular composition or method of administration) to incorporate any of a variety of additives, e.g., components that improve drug delivery, shelf-life and patient acceptance. Suitable additives include acids, antioxidants, antimicrobials, buffers, colorants, crystal growth inhibitors, defoaming agents, diluents, emollients, fillers, flavorings, gelling agents, fragrances, lubricants, propellants, osmotic modifiers, thickeners, salts, solvents, surfactants, other chemical stabilizers, or mixtures thereof. Examples of these additives can be found, for example, in M. Ash and I. Ash, Handbook of Pharmaceutical Additives (Hampshire, England: Gower Publishing, 1995), the contents of which are herein incorporated by reference.

Because of the acid labile nature of certain drugs, and PPIs in general, it may be, as noted above, desirable to incorporate a base into the formulations of the drug to be delivered by the dosage form. Any suitable method for including a base in the formulation may be used. For example, the base may be incorporated into the sub-coating layer of the dosage forms described above with respect to FIGS. 1, 2, 3A-3B. As will be appreciated, effective use of bases can, in some cases, reduce or eliminate the need for enteric coatings. Suitable bases are known in the art, and may include metal and or alkaline salts of carbonates, bicarbonates, hydroxides, and the like. Suitable cations for such salts include aluminum, bismuth, magnesium, calcium, lithium, sodium, potassium, and combinations thereof.

Guidance is provided herein for the administration of the dosage forms of the disclosure. It will be appreciated by the skilled artisan, however, that modifications to dosage, regimen, etc. may be required and is best determined by the practitioner on a patient-by-patient basis. The skilled practitioner will be capable of making such modifications based on commonly available knowledge. The dosage forms are typically employed for once-a-day oral administration.

The formulations described herein may be presented in unit dose form or in multi-dose containers with an optional preservative to increase shelf life. Also contemplated are kits for the treatment of any of the conditions described herein, or any of the conditions that may be treated using the dosage forms described herein. The kit comprises the dosage form in either a single unit container or a multiple unit container, and may further comprise instructions for dosage or administration, package inserts, and the like.

The formulations and dosage forms described herein may be used to treat any condition that would benefit from pulsatile delivery. For example, the materials and methods may be used in the treatment of conditions relating to gastric acid secretion, including GERD and NAB, as well as other diseases, as described in the following section.

### III. METHODS OF TREATMENT AND DRUGS SUITABLE FOR ADMINISTRATION

In a first aspect, a method for treating GERD is provided. Symptoms of gastroesophageal reflux (GER) affect about 45% of the US adult population at least once a month, while 28% experience it at least once weekly and 10% develop heartburn and other symptoms of GER on a daily basis. The weekly and daily refluxers are the patients most likely to be treated with proton pump inhibitors (PPIs). Gastroesophageal reflux disease (GERD) associated with nocturnal acid breakthrough (NAB) while on PPIs or other acid suppressing therapies is a common event. In a recent study NAB was observed in 70% of the GERD patients taking PPIs, while acid exposure to the esophagus (reflux) was observed in 33% of these patients with NAB (Katz P. O. et al., Aliment Pharmacol Ther., 12:1231-4 (1999)). This was confirmed in a study with esomeprazole where only 50% of the GERD patients had relief of nocturnal heartburn. In another study examining various dosing regimens of omeprazole it was found that twice-daily (BID) dosing (20 mg before breakfast and dinner) was most effective for nighttime pH control of the stomach (pH>4 80% of the time), while 40 mg before dinner was intermediate (pH>4 69% of the time), and dosing 40 mg before breakfast (the approved time) was least effective (pH>4 24% of the time). It should be noted that all daytime data were not different between dosing regimens and were minimal. These data indicate that there is an unmet need for control of acid production during the night.

Accordingly, in another aspect, a method for treating, preventing, or reducing the occurrence of NAB is provided. In another aspect, a method for treating GERD and concomitantly treating, preventing, or reducing the occurrence of NAB is provided. In these methods, dosage forms of the type described above are provided, wherein one or more of the pulsed doses released from the dosage form in a PPI. In another embodiment, one of the doses in the dosage form is a PPI, such as omeprazole, and the other dose is a non-steroidal anti-inflammatory agent, such as a salicylate, an arylalkanoic acid, a 2-arylpropionic acid, an N-arylanthranilic acid, a pyrazolidine derivative, an oxicam, or a COX-2 inhibitor. Specifically preferred compounds include, but are not limited to, aspirin, ibuprofen, and naproxen.

Antacids, histamine 2 receptor antagonists (cimetidine, rantidine, famotidine, and nizatidine), and PPI are currently used to treat GERD, although PPIs are generally considered the most efficacious. Omeprazole has no particular advantage over the other PPIs (esomeprazole, lansoprazole, rabeprazole, and pantoprazole) as the efficacy in GERD is the same for all PPIs. In 2005 there were 108 million prescriptions written for oral solid antacids; and of that, 81 million prescriptions were written for PPIs.

As noted above, even when GERD patients are on BID PPIs, NAB occurs in about 20% of the patients. This is likely due to the timing of the evening dose of PPIs, as they are administered before dinner (5-6 μm). When NAB occurs, about 4-6 hr after the evening meal, there is no longer an effective concentration of PPI present because of its short half-life. With the initial dose of a PPI, 60-75% of the proton pumps are inactivated, resulting in 25-40% residual secretion capacity. Additionally, de novo synthesis of new pumps, which occurs mainly at night, adds another 25-30%. With the second day's morning dose, 60-75% of the remaining and regenerated pumps are inhibited. This process continues until a steady state is reached where there is still about a 35% acid secretory capacity. However, as the new pumps are mainly regenerated at night, the pH of the stomach remains high during the day but decreases at night as the new pumps are synthesized and become active (Sachs G., Eur J Gastroenterol Hepatol., 3(Suppl 1):S35-41 (2001)).

While one might assume that nocturnal GER or NAB could be overcome with an extended-release omeprazole formulation, a study indicated a reduced relative bioavailability (61±15%) with a simulated controlled release of omeprazole compared to omeprazole in the fasted state. The reduced bioavailability is likely due to first-pass metabolism, the problematic effects of which are amplified with an extended-release formulation. The dosage forms described herein provide a solution to the problem of NAB that addresses the first-pass metabolism by providing a two pulse system. The unit dose form is taken with dinner, and the first pulse is released immediately after ingestion. This pulse inhibits the proton pumps that are activated by the meal. The second delayed pulse of the formulation is retained in the stomach and releases the second pulse 4-6 hr later, when NAB occurs. This results in an effective concentration of omeprazole being present when the proton pumps become active at night

Thus, in one embodiment, a unit dose form of omeprazole is provided, (in other embodiments, unit dose forms of other PPIs are provided) that yields a delayed pulse and is targeted for patients with GERD, with a specific emphasis on patients who have nocturnal reflux while being treated with PPI. This unit dose form provides a once-daily oral dosage formulation that is administered with the evening meal. In one embodiment, the unit dose form provides a two-pulse delayed release formulation, with 20 mg of omeprazole (or equivalent dose of another PPI) being released immediately and a second 20 mg being released 4-6 hours later. The therapeutic advantage of this unit dose form is that drug will be present when the proton pumps become active during the night, and thus, they would be inhibited.

With the currently marketed delayed release formulations and PPIs' short plasma half-life (0.5-2 hours) when NAB occurs there is no drug remaining in the system to inhibit the proton pumps which become active during this time. A method of treating GERD while preventing or reducing the occurrence of NAB that can be practiced with current marketed delayed release formulations is contemplated. In this embodiment, the patient is administered a dose of 20 mg of omeprazole (or equivalent dose of another PPI) with the evening meal and another dose of at least 20 to 40 mg of omeprazole (or equivalent dose of another PPI) is administered at bed time.

In other embodiments, a multiple unit dosage form is provided, in which the two pulses are delivered in two separate dosage forms. Enteric-coated beads/granules are utilized in both to protect the drug from acid degradation in the stomach. The components for each pulse can be packaged in a single capsule or presented as separate dosage forms (capsule or tablet) under single-unit packaging (blister card). The first pulse is provided by enteric-coated beads/granules or a rapidly disintegrating tablet incorporating enteric-coated beads/granules. The second pulse is a swellable, erodible matrix tablet to ensure the adequate retention in the stomach to deliver the drug 4-6 hours after administration. In another embodiment, a single unit dosage form is provided in which the two-pulse system is delivered in a single unit dosage form, such as a bi-layer or tri-layer tablet. The first active layer delivers the 20 mg of omeprazole immediately after administration. The second active layer (swellable, erodible) delivers another 20 mg 4-6 hours later. Both active layers contain enteric-coated beads/granules of the drug. For the tri-layer tablet, there is a third layer (swellable, erodible) between the two active layers, which is composed of polymer only to provide the gastric retention before the second pulse is delivered.

### A. Omeprazole and Other Proton Pump Inhibitors (PPIs)

In another aspect, methods for administration of therapeutic agents suitable for the treatment of dyspepsia and related conditions, including GERD and other conditions related to the harmful effects of gastric acid secretion in some patients are provided. The active agents suitable for delivery by such methods include, for example, PPIs and H₂-receptor antagonists. These compounds are preferentially absorbed in the upper GI tract and not (or minimally) absorbed in the colon and also are susceptible to substantial first-pass metabolism in the liver.

Proton pump inhibitors suitable to be administered using the methods described herein include those having the structural formula (I), below.

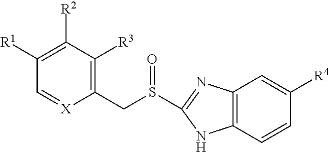

wherein, in formula (I), X is selected from CH and N, and R¹, R², R³, and R⁴ are independently selected from H, C₁-C₁₂ alkyl, and C₁-C₁₂ heteroalkyl. Furthermore, where appropriate, each of R¹, R², R³, and R⁴ may be substituted or unsubstituted, wherein the substituents are selected from halo, C₁-C₁₂ alkyl, partially or fully halogenated C₁-C₁₂ alkyl, C₁-C₁₂ heteroalkyl, and partially or fully halogenated C₁-C₁₂ heteroalkyl. Preferred embodiments of formula (I) include, for example, omeprazole (X═N, R¹═CH₃, R²═OCH₃, R³═CH₃, R⁴═OCH₃), pantoprazole (X═N, R¹═H, R²═OCH₃, R³═OCH₃, R⁴═OCHF₂), lansoprazole (X═N, R¹═H, R²═OCH₂CF₃, R³═CH₃, R⁴═H), rabeprazole (X═N, R¹═H, R²═OCH₂CH₂CH₂OCH₃, R³═CH₃, R⁴═H), and leminoprazole (X═CH, R¹═H, R²═H, R³═N(CH₃)CH₂CH(CH₃)₂, R⁴═H). Single enantiomers (such as esomeprazole), as well as racemic mixtures of the compounds having the structure of formula (I) are also within the scope of this disclosure. Moreover, PPIs of other structure, including related structures, such as that of tenatoprazole, and PPIs of unrelated structure, are within the scope of this disclosure. See also U.S. Pat. No. 5,753,265, incorporated herein by reference, for other compounds that may be incorporated into the dosage forms described herein. More generally, the dosage forms are applicable to any drug that undergoes first-pass metabolism and is poorly absorbed in the colon (such as H₂ antagonists).

Proton pump inhibitors (PPIs) have become one of the most commonly prescribed classes of medications in the primary care setting. Since their introduction in the late 1980's, PPIs have improved treatment of various acid-peptic disorders, including gastroesophageal reflux disease (GERD), peptic ulcer disease and nonsteroidal anti-inflammatory drug-induced gastropathy. Use of PPIs in the treatment of patients who suffer from gastric acid-related disorders has led to increased quality of life, productivity, and overall well-being of these patients.

Proton pump inhibitors provide symptomatic relief of heartburn associated with GERD by suppressing gastric acid, causing an increase in the pH of the refluxate. Thus, the outcome of pharmacologic therapy of GERD is dependent upon the acid-inhibitory effectiveness of the agents. Omeprazole, a compound of the substituted benzimidazole class, inhibits gastric acid secretion. The mechanism of action of omeprazole is to selectively inhibit the parietal cell membrane enzyme (H+, K+)-ATPase, the “proton pump.” Results from studies in healthy volunteers and patients have shown that omeprazole administered in a dose of 20 mg provides a 78% decrease in basal acid output 2-6 hours after dosing and a 50%-80% decrease in basal acid output 24 hours after dosing.

Omeprazole is approved for marketing in the United States for short-term treatment of active duodenal ulcer, short-term treatment of active benign gastric ulcer, short-term treatment of erosive esophagitis; treatment of heartburn and other symptoms associated with GERD; maintenance of healing of erosive esophagitis; long-term treatment of pathological hypersecretory conditions; and treatment of patients with H. pylori and duodenal ulcer disease in combination with clarithromycin or with clarithromycin and amoxicillin.

Some patients with symptomatic GERD are partially responsive to PPI therapy in that they experience few or no symptoms during the day but suffer from nocturnal heartburn. Because the decrease in basal acid output is dependent on time since dosing, these partially responsive patients should benefit from the alternative dosing regimens provided herein. Specifically, a method for treating GERD in a patient is provided, the method comprising administering to the patient a single unit dose that provides a two-pulse regimen of omeprazole or another PPI, in which the dosage form is administered contemporaneously with dinner and the first pulse is released shortly after ingestion of the dosage form and the second pulse is released 4 to 6 to 8 or more hours after ingestion of the dosage form. Typically, each pulse of omeprazole will be about 20 mg, and administration of this dosage form should reduce the occurrence of nocturnal acid breakthrough and nocturnal acid reflux compared to alternate dosing regimens, such as the administration of 40 mg of omeprazole taken 30 to 60 minutes prior to dinner. The benefits of this dosing method in clinical studies are described in Example 1, below.

For the treatment of GERD, the pulsatile dosage forms disclosed herein allow for once-a-day administration. For example, a patient desiring treatment may take a pulsatile dosage form once daily with the evening meal. The initial (i.e., immediate release) pulse provides a pharmaceutically effective amount of the active agent to control gastric acid secretion during and immediately after the evening meal. The delayed pulse then provides a pharmaceutically effective amount of the active agent during the night, thereby helping to maintain gastric acid secretion at night. The delayed pulse therefore treats GERD and helps to prevent or suppress NAB. In general, the maximal benefit from PPI therapy is achieved when PPIs are taken 15-30 minutes before meals, allowing optimal blood concentration of the drug at the time of meal-induced activation of proton pumps, and the influence of a large number of pumps. In one embodiment, the active agent is omeprazole and the total dose of omeprazole in each dosage form is between about 1 mg and 500 mg, or between about 10 mg and 80 mg.

Omeprazole is not or only minimally absorbed in the colon. In addition, the first-pass metabolism is so great that bioavailability is substantially reduced in conventional extended-release dosage forms. Accordingly, the dosage forms described herein are designed to provide pulsatile delivery of active agent in the upper GI tract. Preferably, the active agent is protected by an enteric coating while in the stomach and/or until just after leaving the stomach, where it is released in the duodenum and small intestines.

Specifically, a gastric retentive dosage form is preferred. The gastric retentive characteristics are based on the size of the tablet or particles in the presence of food. Gastric retention is achieved by having a dosage form that is either sufficiently large initially or swells to a size that promotes retention. Swelling can be achieved by the use of hydrophilic polymers such as polyethylene oxide or HPMC and may, but need not, also include gas-generating agents to promote swelling or increase buoyancy.

Optionally, the dosage form releases an initial pulse of acid-protected omeprazole in the form of particles (e.g., beads or pellets). Acid protection results either from an enteric or delayed release coating or by including a base in the initial release formulation. Generally, the initial pulse provides an immediate release of active agent, and any appropriate method for the immediate-release administration of PPIs may be used. The acid labile nature of omeprazole and other PPIs must be considered when formulating the first pulse. As will be appreciated by the skilled artisan, a number of different methods may be employed to obtain the initial (i.e., immediate) pulse of active agent. The dosage forms described in the preceding section and in the examples below are ideally suited for the administration of omeprazole and other PPIs for the treatment of GERD and preventing or reducing the frequency of occurrence of NAB.

### B. Other Drugs

It will be recognized by those of skill in the art that the methods of administration and dosage forms described herein are also suitable for therapeutic agents other than PPIs, including drugs and active agents that are suitable for treatment of conditions other than GERD and related conditions. Such therapeutic agents include those commonly administered via the oral route, those where oral administration is desirable, and those that have not previously been administered via the oral route but that would benefit from delivery via the oral route using the methods and dosage forms described herein.

In one embodiment, the dosage forms described herein find use for drugs that have a reduced absorption in the lower GI tract and a reduced bioavailability due to first-pass metabolism. Sparingly soluble drugs particularly can suffer from both of these absorption issues, since hepatic metabolism tries to make these sparingly soluble drugs more polar to eliminate them vial renal clearance, and the drug's poor solubility makes the upper GI tract too short for adequate absorption. Any of the drugs in the examples listed below that are sparingly soluble are contemplated to benefit from administration in a dosage form as described herein.

Active agents for use in the dosage forms described herein may include anti-microbial agents, anti-diabetic agents, analgesics, anti-inflammatory agents, anti-convulsant agents, CNS and respiratory stimulants, neuroleptic agents, hypnotic agents and sedatives, anxiolytics and tranquilizers, other anti-cancer drugs including antineoplastic agents, antihyperlipidemic agents, antihypertensive agents, cardiovascular preparations, anti-viral agents, sex steroids, muscarinic receptor agonists and antagonists, and macromolecular active agents such as DNA, RNA, proteins, and peptide drugs. Some examples of these active agents are provided below.

Analgesics useful in the dosage forms described herein include by way of example non-opioid analgesic agents such as apazone, etodolac, difenpiramide, indomethacin, meclofenamate, mefenamic acid, oxaprozin, phenylbutazone, piroxicam, and tolmetin; and opioid analgesics such as alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol. Additional analgesic agents contemplated for use in the dosage forms described herein include non-steroidal anti-inflammatory agents (NSAIDs). Examples of suitable commercially available opioid analgesics useful in the dosage forms include PERCOCET® (oxycodone; Dupont Merck Pharmaceuticals, Wilmington, Del.), ULTRACET® (tramadol; Johnson & Johnson, New Brunswick, N.J.), and CLONOPIN™ (clonazepam; Hoffmann-LaRoche, Nutley, N.J.). It will be appreciated that combinations of analgesic agents can be used in a single dosage form, for example, an opioid analgesic in combination with a non-opioid analgesic. Combinations of hydrocodone or hydromorphone and ibuprofen or acetaminophen are exemplary of such combinations.

Anti-cancer agents, including antineoplastic agents useful in the dosage forms include by way of example paclitaxel, docetaxel, camptothecin and its analogues and derivatives (e.g., 9-aminocamptothecin, 9-nitrocamptothecin, 10-hydroxy-camptothecin, irinotecan, topotecan, 20-O-β-glucopyranosyl camptothecin), taxanes (baccatins, cephalomannine and their derivatives), carboplatin, cisplatin, interferon-α_{2A}, interferon-α_{2B}, interferon-α_{N3} and other agents of the interferon family, levamisole, altretamine, cladribine, tretinoin, procarbazine, dacarbazine, gemcitabine, mitotane, asparaginase, porfimer, mesna, amifostine, mitotic inhibitors including podophyllotoxin derivatives such as teniposide and etoposide and vinca alkaloids such as vinorelbine, vincristine and vinblastine.

Anti-convulsant (anti-seizure) agents useful in the dosage forms include by way of example azetazolamide, carbamazepine, clonazepam, clorazepate, ethosuximide, ethotoin, felbamate, lamotrigine, mephenytoin, mephobarbital, phenytoin, phenobarbital, primidone, trimethadione, vigabatrin, topiramate, and the benzodiazepines. Benzodiazepines, as is well known, are useful for a number of indications, including anxiety, insomnia, and nausea. Examples of suitable commercially available anti-convulsants useful in the dosage forms of include TEGRETOL® (carbamazepine; Novartis, Summit, N.J.), DILANTIN® (Pfizer Inc., New York, N.Y.) and LAMICTAL® (lamotrigine (GlaxoSmithKline, Philadelphia, Pa.).

Anti-depressant agents useful in the dosage forms include by way of example the tricyclic antidepressants LIMBITROL® (amitriptyline; Hoffmann-LaRoche, Nutley, N.J.), TOFRANIL® (imipramine; Tyco Healthcare, Mansfield, Mass.), ANAFRANIL™ (clomipramine; Tyco Healthcare, Mansfield, Mass.), and NORPRAMIN® (desipramine; Sanofi-Aventis, Bridgewater, N.J.).

Anti-diabetic agents useful in the dosage forms include by way of example acetohexamide, chlorpropamide, ciglitazone, gliclazide, glipizide, glucagon, glyburide, miglitol, pioglitazone, tolazamide, tolbutamide, triampterine, and troglitazone.

Anti-hyperlipidemic agents useful in the dosage forms include by way of example lipid-lowering agents, or “hyperlipidemic” agents, such as HMG-CoA reductase inhibitors such as atorvastatin, simvastatin, pravastatin, lovastatin and cerivastatin, and other lipid-lowering agents such as clofibrate, fenofibrate, gemfibrozil and tacrine.

Anti-hypertensive agents useful in the dosage forms include by way of example amlodipine, benazepril, darodipine, diltiazem, doxazosin, enalapril, eposartan, esmolol, felodipine, fenoldopam, fosinopril, guanabenz, guanadrel, guanethidine, guanfacine, hydralazine, losartan, metyrosine, minoxidil, nicardipine, nifedipine, nisoldipine, phenoxybenzamine, prazosin, quinapril, reserpine, terazosin, and valsartan.

Anti-inflammatory agents useful in the dosage forms include by way of example nonsteroidal anti-inflammatory agents such as the propionic acid derivatives as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, and fenbufen; apazone; diclofenac; difenpiramide; diflunisal; etodolac; indomethacin; ketorolac; meclofenamate; nabumetone; phenylbutazone; piroxicam; sulindac; and tolmetin, and steroidal anti-inflammatory agents such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, and methylprednisolone.

Anti-microbial agents useful in the dosage forms include by way of example tetracycline antibiotics and related compounds (chlortetracycline, oxytetracycline, demeclocycline, methacycline, doxycycline, minocycline, rolitetracycline); macrolide antibiotics such as erythromycin, clarithromycin, and azithromycin; streptogramin antibiotics such as quinupristin and dalfopristin; beta-lactam antibiotics, including penicillins (e.g., penicillin G, penicillin VK), antistaphylococcal penicillins (e.g., cloxacillin, dicloxacillin, nafcillin, and oxacillin), extended spectrum penicillins (e.g., aminopenicillins such as ampicillin and amoxicillin, and the antipseudomonal penicillins such as carbenicillin), and cephalosporins (e.g., cefadroxil, cefepime, cephalexin, cefazolin, cefoxitin, cefotetan, cefuroxime, cefotaxime, ceftazidime, and ceftriaxone), and carbapenems such as imipenem, meropenem and aztreonam; aminoglycoside antibiotics such as streptomycin, gentamicin, tobramycin, amikacin, and neomycin; glycopeptide antibiotics such as teicoplanin; sulfonamide antibiotics such as sulfacetamide, sulfabenzamide, sulfadiazine, sulfadoxine, sulfamerazine, sulfamethazine, sulfamethizole, and sulfamethoxazole; quinolone antibiotics such as ciprofloxacin, nalidixic acid, and ofloxacin; anti-mycobacterials such as isoniazid, rifampin, rifabutin, ethambutol, pyrazinamide, ethionamide, aminosalicylic, and cycloserine; systemic antifungal agents such as itraconazole, ketoconazole, fluconazole, and amphotericin B; antiviral agents such as acyclovir, famcicylovir, ganciclovir, idoxuridine, sorivudine, trifluridine, valacyclovir, vidarabine, didanosine, stavudine, zalcitabine, zidovudine, amantadine, interferon alpha, ribavirin and rimantadine; and miscellaneous antimicrobial agents such as chloramphenicol, spectinomycin, polymyxin B (colistin), bacitracin, nitrofurantoin, methenamine mandelate and methenamine hippurate.

Anti-viral agents useful in the dosage forms include by way of example the antiherpes agents acyclovir, famciclovir, foscarnet, ganciclovir, idoxuridine, sorivudine, trifluridine, valacyclovir, and vidarabine; the antiretroviral agents didanosine, stavudine, zalcitabine, and zidovudine; and other antiviral agents such as amantadine, interferon alpha, ribavirin and rimantadine.

Anxiolytics and tranquilizers useful in the dosage forms include by way of example benzodiazepines (e.g., alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, triazolam), buspirone, chlordiazepoxide, and droperidol.

Cardiac agents, which can be used in combination with diuretics, useful in the dosage forms include by way of example amiodarone, amlodipine, atenolol, bepridil, bisoprolol bretylium, captopril, carvedilol, diltiazem, disopyramide, dofetilide, enalaprilat, enalapril, encainide, esmolol, flecainide, fosinopril, ibutilide, inamrinone, irbesartan, lidocaine, lisinopril, losartan, metroprolol, nadolol, nicardipine, nifedipine, procainamide, propafenone, propranolol, quinapril, quinidine, ramipril, trandolapril, and verapamil.

Cardiovascular agents useful in the dosage forms include by way of example angiotensin converting enzyme (ACE) inhibitors, cardiac glycosides, calcium channel blockers, beta-blockers, antiarrhythmics, cardioprotective agents, and angiotensin II receptor blocking agents. Examples of the foregoing classes of drugs include the following: ACE inhibitors such as enalapril, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-benzazepine-1-acetic acid or 3-(1-ethoxycarbonyl-3-phenyl-(1S)-propylamino)-2,3,4,5-tetrahydro-2-oxo-(3S)-benzazepine-1-acetic acid monohydrochloride; cardiac glycosides such as digoxin and digitoxin; inotropes such as amrinone and milrinone; calcium channel blockers such as verapamil, nifedipine, nicardipene, felodipine, isradipine, nimodipine, bepridil, amlodipine and diltiazem; beta-blockers such as atenolol, metoprolol; pindolol, propafenone, propranolol, esmolol, sotalol, timolol, and acebutolol; antiarrhythmics such as moricizine, ibutilide, procainamide, quinidine, disopyramide, lidocaine, phenytoin, tocainide, mexiletine, flecainide, encainide, bretylium and amiodarone; and cardioprotective agents such as dexrazoxane and leucovorin; vasodilators such as nitroglycerin; and angiotensin II receptor blocking agents such as losartan, hydrochlorothiazide, irbesartan, candesartan, telmisartan, eposartan, and valsartan.

CNS and respiratory stimulants useful in the dosage forms include by way of example xanthines such as caffeine and theophylline; amphetamines such as amphetamine, benzphetamine hydrochloride, dextroamphetamine, dextroamphetamine sulfate, levamphetamine, levamphetamine hydrochloride, methamphetamine, and methamphetamine hydrochloride; and miscellaneous stimulants such as methylphenidate, methylphenidate hydrochloride, modafinil, pemoline, sibutramine, and sibutramine hydrochloride.

Hypnotic agents and sedatives useful in the dosage forms include by way of example clomethiazole, ethinamate, etomidate, glutethimide, meprobamate, methyprylon, zolpidem, and barbiturates (e.g., amobarbital, apropbarbital, butabarbital, butalbital, mephobarbital, methohexital, pentobarbital, phenobarbital, secobarbital, thiopental).

Muscarinic receptor agonists and antagonists useful in the dosage forms include by way of example choline esters such as acetylcholine, methacholine, carbachol, bethanechol (carbamylmethylcholine), bethanechol chloride, cholinomimetic natural alkaloids and synthetic analogs thereof, including pilocarpine, muscarine, McN-A-343, and oxotremorine. Muscarinic receptor antagonists are generally belladonna alkaloids or semisynthetic or synthetic analogs thereof, such as atropine, scopolamine, homatropine, homatropine methyl bromide, ipratropium, methantheline, methscopolamine and tiotropium.

Neuroleptic agents useful in the dosage forms include by way of example antidepressant drugs, antimanic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, fluoxetine, fluvoxamine, paroxetine, sertraline, and venlafaxine, (c) monoamine oxidase inhibitors such as phenelzine, tranylcypromine, and (−)-selegiline, and (d) other, “atypical” antidepressants such as nefazodone, trazodone and venlafaxine, and wherein antimanic and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole.

Peptide drugs useful in the dosage forms include by way of example the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP),and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin. Still other peptidyl drugs that can be advantageously delivered using the present systems include endorphins (e.g., dermorphin, dynorphin, α-endorphin, β-endorphin, γ-endorphin, α-endorphin, [Leu⁵]enkephalin, [Met⁵]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof.

Sex steroids useful in the dosage forms include by way of example progestogens such as acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. Also included within this general class are estrogens, e.g.: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. Androgenic agents, also included within the general class of sex steroids, are drugs such as the naturally occurring androgens androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, dehydroepiandrosterone (DHEA; also termed “prasterone”), sodium dehydroepiandrosterone sulfate, 4-dihydrotestosterone (DHT; also termed “stanolone”), 5α-dihydrotestosterone, dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol and testosterone; pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters; and pharmaceutically acceptable derivatives of testosterone such as methyl testosterone, testolactone, oxymetholone and fluoxymesterone.

Where appropriate, any of the active agents described herein may be administered in the form of a salt, ester, amide, prodrug, conjugate, active metabolite, isomer, fragment, analog, or the like, provided that the salt, ester, amide, prodrug, conjugate, active metabolite, isomer, fragment, or analog is pharmaceutically acceptable and pharmacologically active in the present context. Salts, esters, amides, prodrugs, conjugates, active metabolites, isomers, fragments, and analogs of the agents may be prepared using standard procedures known to those skilled in the art of synthetic organic chemistry and described, for example, by J. March, Advanced Organic Chemistry: Reactions, Mechanisms and Structure, 5th Edition (New York: Wiley-Interscience, 2001). For example, where appropriate, any of the compounds described herein may be in the form of a prodrug. The prodrug requires conversion to the active agent. Such conversion may involve, for example, protonation by an acid. Most PPIs are prodrugs that are converted to an active form in the acid environment of the canaliculi after being secreted by the parietal cells.

Where appropriate, any of the compounds described herein may be in the form of a pharmaceutically acceptable salt. A pharmaceutically acceptable salt may be prepared from any pharmaceutically acceptable organic acid or base, any pharmaceutically acceptable inorganic acid or base, or combinations thereof. The acid or base used to prepare the salt may be naturally occurring.

Suitable organic acids for preparing acid addition salts include, e.g., C₁-C₆ alkyl and C₆-C₁₂ aryl carboxylic acids, di-carboxylic acids, and tri-carboxylic acids such as acetic acid, propionic acid, succinic acid, maleic acid, fumaric acid, tartaric acid, glycolic acid, citric acid, pyruvic acid, oxalic acid, malic acid, malonic acid, benzoic acid, cinnamic acid, mandelic acid, salicylic acid, phthalic acid, and terephthalic acid, and aryl and alkyl sulfonic acids such as methanesulfonic acid, ethanesulfonic acid, and p-toluenesulfonic acid, and the like. Suitable inorganic acids for preparing acid addition salts include, e.g., hydrochloric acid, hydrobromic acid, hydroiodic acid, sulfuric acid, nitric acid, and phosphoric acid, and the like. An acid addition salt may be reconverted to the free base by treatment with a suitable base.

Suitable organic bases for preparing basic addition salts include, e.g., primary, secondary and tertiary amines, such as trimethylamine, triethylamine, tripropylamine, N,N-dibenzylethylenediamine, 2-dimethylaminoethanol, ethanolamine, ethylenediamine, glucamine, glucosamine, histidine, and polyamine resins, cyclic amines such as caffeine, N-ethylmorpholine, N-ethylpiperidine, and purine, and salts of amines such as betaine, choline, and procaine, and the like. Suitable inorganic bases for preparing basic addition salts include, e.g., salts derived from sodium, potassium, ammonium, calcium, ferric, ferrous, aluminum, lithium, magnesium, or zinc such as sodium hydroxide, potassium hydroxide, calcium carbonate, sodium carbonate, and potassium carbonate, and the like. A basic addition salt may be reconverted to the free acid by treatment with a suitable acid.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

Any of the compounds described herein may be the active agent in a formulation as described herein. Formulations may include one, two, three, or more than three of the active agents and drugs described herein, and may also include one or more active agents not specifically recited herein.

When a dosage form or method is used or practiced in combination with the administration of another agent, such as secondary analgesics, anticonvulsant agents, antidepressants, and the like, the additional agent may be obtained from a commercial source in a variety of dosage forms (e.g., tablets, capsules, oral suspensions, and syrups). The additional agent may be administered as a separate dosage form or a gastric retentive dosage form of the present invention may comprising the additional agent may be used.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### IV. EXAMPLES

The following examples are illustrative in nature and are in no way intended to be limiting.

### Example 1

### Method of Treating GERD and Preventing or Reducing NAB

A study was conducted to demonstrate the limited colonic absorption of omeprazole. Nine healthy subjects were entered into the study with an intention to complete treatment of at least six subjects. The study was a four-way crossover study with the following doses administered: (i) simulated control release (SCR): 20 mg omeprazole divided into 17 doses, administered at 30 minute intervals (8 hr of delivery), in the fed state; (ii) 20 mg omeprazole in the fed state; (iii) 20 mg omeprazole in the fasted state; and (iv) 20 mg omeprazole delivered to the ascending colon via the ENTERION™ capsule (radio controlled capsule to control release of drug; the position in the GI tract is determined by scintigraphy). A period of at least four days for washout was allowed between dosing.

Seven subjects completed the study. Table 1 lists the mean±SD of the pharmacokinetic parameters determined from the blood plasma drug concentrations from blood samples taken during the dosing period.

TABLE 1
Omeprazole Pharmacokinetic Parameters (n = 7)

| | Dosing Arm | | | |
|---|---|---|---|---|
| | (i) SCR | (ii) Fed | (iv) Colonic | (iii) Fasted |
| AUC | 568 ± 727* | 849 ± 999 | 242 ± 323* | 969 ± 1086 |
| (ng · hr/mL) | | | | |
| Relative | 61 ± 15* | 90 ± 16 | 22 ± 12* | 100 |
| bioavailability | | | | |
| (%) | | | | |
| Cmax | 111 ± 92 | 296 ± 256 | 47 ± 40 | 408 ± 288 |
| (ng/mL) | | | | |
| Relative Cmax | 28 ± 10 | 72 ± 36 | 10 ± 5 | 100 |
| (%) | | | | |
| Tmax | 4.9 | 3.7 | 1.8 | 1.5 |

*= p < 0.05 compared to fasting

As seen in Table 1, there was a statistically significant reduction in bioavailability of omeprazole when delivered to the colon or by SCR compared to patients taking the drug dose when in a fasted state. In contrast, when omeprazole was dosed to patients in the fed state, there was no statistically significant difference compared to the fasted state. In fact, if one of the subjects was removed from the analysis, then the relative bioavailability in the fed state was 96±7% compared to fasting. The ENTERION capsule was activated in the terminal ileum in one of the subjects, so this subject was not included in colonic absorption study parameters. However the relative bioavailability compared to fasted state was 58% in this subject, indicating good absorption of omeprazole in the terminal ileum.

The results of this study show that omeprazole is not substantially absorbed in the colon, so delivery of omeprazole should be targeted to the small intestine. Administration of omeprazole in a controlled release regimen, as achieved in the SCR dosing arm, reduced bioavailability. This is likely due to first-pass metabolism, indicating that a sustained-release formulation of omeprazole is unlikely to provide adequate levels to inhibit NAB. Fasting and fed pharmacokinetic parameters were not significantly different, indicating omeprazole can be given in either state. The data are supportive of the conclusion that NAB can be prevented with a dosage form that provides a two-pulse delivery of omeprazole. Such a dosage form is preferably taken with dinner and the first pulse is released immediately. This pulse would inhibit the proton pumps that are activated by the meal. All or a portion of the dosage form would be retained in the stomach for release of a second pulse 4-6 hours after ingestion of the dosage form. Thus, when NAB occurs, an effective concentration of omeprazole is provided at a time when the proton pumps become active at night.

### Example 2

### Method of Treating GERD and/or NAB

A randomized, open-label, two-period crossover study in GERD patients between 18 and 65 years of age, inclusive, with nocturnal reflux after receiving PPIs for at least 3 months, was conducted to demonstrate the efficacy of a two-pulse dosing regimen for treating GERD and/or NAB. Sixteen patients with a history of GERD, all of whom experienced recurrent nighttime reflux for at least three months while taking proton pump inhibitors, were enrolled. The study was an open label crossover study in which 14 of the 16 patients participated in each of two treatment arms separated by a washout period. In one treatment arm, the patients received 40 mg of omeprazole 30 minutes before dinner, for six days. In the other treatment arm, the patients received 20 mg of omeprazole at dinner followed by an additional 20 mg of omeprazole four hours later, for six days. Ambulatory 24-hour gastric pH was recorded and blood samples taken for PK analysis on days 6-7. Following a seven day washout the patients were crossed over to the alternate treatment. NAB was defined as an intra-gastric pH<4 for more than 1 hour between 22:00 hour and 06:00 hour (10:00 PM and 6:00 AM).

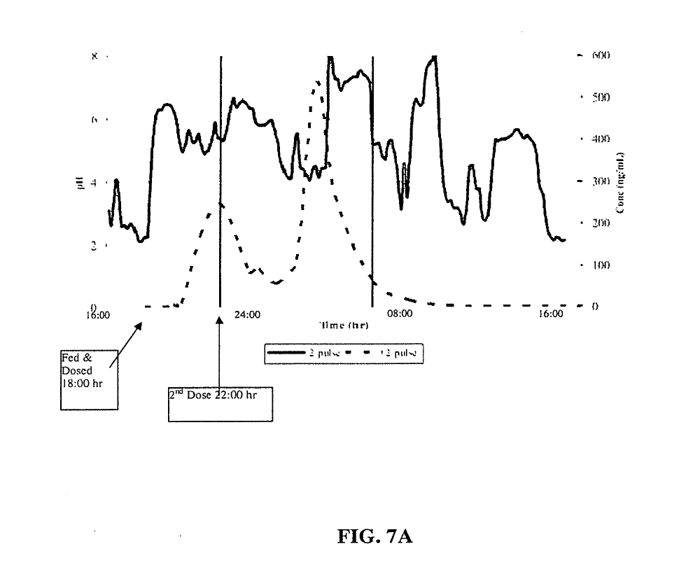

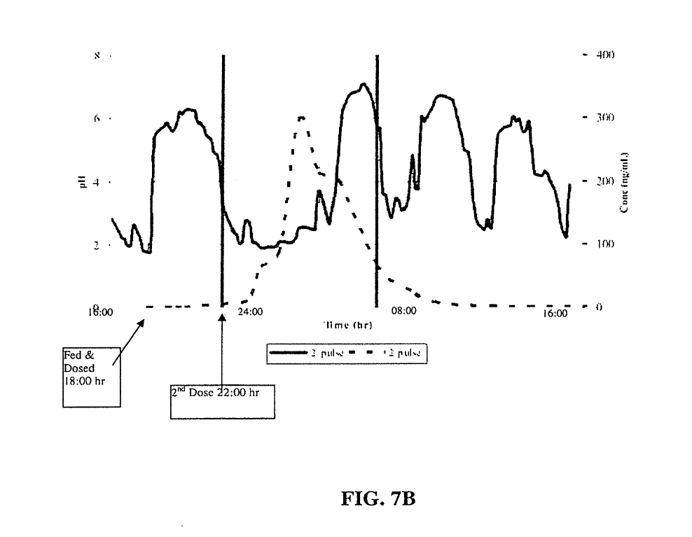

Blood samples taken from the patients were analyzed for omeprazole concentration. The data showed that 9 of the 14 patients who completed the two dose arm of the study began absorbing the first 20 mg dose of omeprazole promptly following ingestion of the drug. These 9 patients also demonstrated an omeprazole absorption profile consistent with the administration of two doses of omeprazole 4 hours apart, as seen in FIG. 7A. All 9 (100%) of the patients experienced inhibition of NAB.

Five (36%) of the 14 patients did not start absorbing omeprazole until 4-5 hours after the initial 20 mg dose was administrated, as seen in FIG. 7B. All five of those patients experienced NAB. Since the exposure to omeprazole as determined by plasma omeprazole area under the curve (AUC), shown in Table 2 below, was equivalent in the patients with the absorption profile of FIG. 7A and the absorption profile of FIG. 7B, it would appear that there was a delay in emptying of the omeprazole pellets in the latter patient group, i.e., in the five subjects that experienced NAB. Indeed, a recent study has show that about 40% of GERD patient have delayed gastric emptying (Neurogastroenterol Motil, 18:894 (2006)), a percentage similar to what was observed in the patients who didn't demonstrate a two pulse PK profile (36%).

TABLE 2

| | median AUC (25-75%) |
|---|---|
| | (ng · hr/mL) |
| Parameter | n = 14 |
| AUC (ng · hr/mL) (All) | 1864 (1299-3167) |
| AUC (with NAB) (n = 5) | 1674 (1211-2607) |
| AUC (without NAB) (n = 9) | 1912 (1299-3167) |

In the 40 mg single dose treatment arm, all 14 patients who completed the study began absorbing the single 40 mg dose promptly following ingestion. Three of the patients experienced NAB.

In summary, all nine subjects that demonstrated a two pulse absorption profile did not experience NAB. Therefore, omeprazole delivered as a two pulsed doses, one dose at dinner and a second dose 4-6 hours later, controls acid reflux and resulting NAB. Omeprazole pellets have a mean±SD diameter of 1.3±0.1 mm. In subjects with delayed gastric emptying this size would be retained until most of the meal has emptied. Thus in order to deliver two pulses in GERD patients with delayed gastric emptying the omeprazole beads having a diameter of 0.5-0.7 mm are preferred.

The objective of the study was to determine if the delivery of a dose of omeprazole with dinner and a second dose four hours after dinner would reduce the incidence of NAB, which typically occurs in the late evening and early morning hours. In the first treatment arm, patients received 20 mg of omeprazole with dinner followed by a second 20 mg dose 4 hours later, in order to simulate a two pulse delivery mechanism. Nine of these patients achieved blood levels from both doses of omeprazole, and thus provided useful data for this two pulse proof of concept trial, none experienced NAB. In the comparative arm of the study, patients received 40 mg of omeprazole 30 minutes before dinner. In this treatment group, three patients experienced NAB, and all three of them had blood levels of omeprazole fall to undetectable levels between 2:00 and 3:00 AM. Results from both arms of the study therefore demonstrate the need to maintain adequate blood levels of omeprazole to inhibit NAB.

A gastric retentive formulation of the S-enantiomer of omeprazole (esomeprazole) can predictably deliver omeprazole approximately four hours after ingestion. Thus, a method of treating GERD while preventing NAB is contemplated. In one embodiment, the method can be practiced by administering to the patient an immediate release dosage form, such as PRILOSEC which contains esomeprazle, or an equivalent dose form of another PPI, contemporaneously with the evening meal and administering a gastric retentive form of the S-enantiomer of omeprazole or an equivalent PPI at bedtime. In another embodiment, the method is practiced by administering a dosage form contemporaneously with the evening meal that provides two pulses of release, one that protects from GERD during and after the evening meal, and the other that is delivered to the stomach at a time removed from ingestion of the dosage form to protect from NAB during the night.

### Example 3

### Shell and Core Tablet

In one embodiment, a dosage form that provides a delayed pulse of drug release created by a core tablet or pellet containing the drug that is surrounded by a coating or shell such that the dosage form releases the drug in a pulse (optionally, the drug is an acid-protected PPI; the acid-protected PPI can be an enteric or delayed release coated particle, bead or pellet or alternatively a particle bead or pellet containing base) after a delay (relative to the time of ingestion) is provided. This dosage form can be referred to as a “press coated” tablet or a “shell and core” tablet. This example describes a dosage form comprising a drug-containing core surrounded by an erodible, swellable, layer designed to promote gastric retention and retard the release of a drug for a pre-selected period of time, between about 1 and 12 hours. If the drug in the dosage form is omeprazole or another acid labile drug, then the drug-containing particle can be protected from the acidic conditions present in the stomach with an enteric protective polymeric coating. In the dosage form illustrated in this example, the drug containing core releases the drug immediately (in an immediate release (IR) fashion) following erosion of the erodible, swellable coating, and the drug is then released from the stomach soon after this immediate release burst from the dosage form by employing a plurality of drug-containing, enteric coated beads, such as the beads described with respect to FIG. 2 above, compressed into a core tablet in a matrix of pharmaceutical excipients.

It is also contemplated to provide a dosage form can deliver a drug in a typical, sustained-release mode, in addition to the pulsatile delivery, by incorporating drug into the core along with the, swellable, erodible polymer. As the shell swells, drug diffuses out of the shell, or is released as the polymer erodes, depending on the aqueous solubility of the drug.

In tests comparing the acid resistance of uncompressed omeprazole-containing beads to those which had been compressed into a core, using formulations containing polyol excipients selected for their ability to bring water into the dosage form, specifically Xylitab 300 (granulated Xylitol, Danisco A/S, Copenhagen, Denmark), higher drug loss (as tested by a derivation of the acid resistance test listed in the USP monograph for omeprazole delayed release capsules) was observed for formulations that had been compressed into core tablets than those that were never compressed. These tests indicated that some enteric protection was lost during compression. While this loss was not complete and the compressed forms could still be employed for the intended purpose, subsequent work focused on finding a blend of excipients that (1) protect the enteric coating on the beads from cracking upon core tablet compression, (2) supply suitable hardness (optimal minimum of 3 kilopons (kp)) (3) demonstrate immediate release as determined using a USP disintegration tester (tablet dissolved in less than 30 minutes), and (4) do not cause cracking of the erodible, swellable shell upon the compression of the shell onto the core.

Tablets were made using typical tablet compression tooling, such as that supplied by Natoli Engineering of Saint Charles, Mo., and compressed using a typical tablet press, such as the Carver Autopress C (Fred Carver, Inc. Wabash, Ind.). Initial work focused on the polymer Polyox (polyethylene oxide, Dow Chemicals, Midland, Mich.) surrounding the core. This work required a tooling set of the same shape as the core, but larger by 2-4 mm in all sides to allow a 1-2 mm thick shell on all sides. Initial work focused on high molecular weight (MW) Polyox, i.e. Polyox WSR 303 in a thin (1 mm) layer around the core, which was centered to maintain a I mm layer to provide the delay of drug release. These core and shell tablets were tested for drug release using a USP apparatus III tester.

Thus, core excipients such as polyethylene glycol and polyethylene oxides, in high concentration, retard disintegration (DS) time. Additives such as superdisintegrants, i.e., Polyplasdone XL (crospovidone, USP by International Specialty Products Corporation, Wayne, N.J.), polyols, sugars, and diluents, reduce DS time. Some of these excipients reduce the hardness, and binders (such as Plasdone K29/32 (povidone, USP by ISP)) can be added to increase hardness.

A thin layer of high MW polymer provided a delay in release, but the drug was released in an abbreviated, controlled-release fashion after that, taking 1-2 hours for the omeprazole to be released after the delay. An optimal omeprazole release for omeprazole is about 30 minutes. Reducing the molecular weight of the polymer modulated both the delay, and the rate of drug release following delay, but did not provide an optimal IR burst. Additives to the polymer, including lactose, polyplasdone, and polyols, i.e. PEARLITOL 300 DC (mannitol USP, Roquette, Lestrem, France), improved the immediate release (IR) burst characteristics. Optimal thickness of the shell layer is 1.6 mm surrounding the core, meaning that the shell's width is a total of 3.2 mm wider than the core.

In particular, the polyols proved very effective in promoting an IR burst following the delay provided by the polymer. Hydration, swelling, and erosion of poly(ethylene oxide) (POLYOX™) occurs on the hydration front as water penetrates the monolithic polymer matrix, which may contribute to the observed controlled release burst using shells with the high molecular weight poly(ethylene oxide) with no additives. The addition of polyols, with their high osmotic potential, can expedite this hydration, swelling, erosion process such that this process occurs within the polymer all at once, as opposed to in sequential nature typical in polymer monoliths, due to the enhanced water penetration. This allows for the catastrophic failure of the shell, following the appropriate delay, promoting the desired IR burst of the core's contents from the shell and core dosage form.

Beads were prepared as follows: sugar spheres from NP Pharm size 355-425 μm coated with (in order): (1) omeprazole coat: 87.1% omeprazole, 12.2% hydroxypropyl methylcellulose, 0.7% TWEEN 80; (2) subcoat: OPADRY Clear YS-1-19025-A; and (3) enteric coat: 80.4% EUDRAGIT L30D55, 16,6% PlasACRYL, 2.9% triethyl citrate.

The dosage form core was prepared from the beads as follows. Beads were cogranulated with a blend that is 30% beads, 59.5% Carbowax (polyethylene glycol), 7% Xylitab 300 (xylitol), 3.5% Povidone K29/32 (povidone). 250 mg of the blend was tableted with a flat faced round, beveled edge tool 0.3236″ diameter.

The shell was prepared from a blend of 70% Polyox 1105 LEO NF grade (polyethylene oxide), 29.5% Pearlitol 300 DC (mannitol), and 0.5% mg stearate. 500 mg was compressed around the core, which was centered in the tablet. Tooling was a 0.4500″ deep concave.

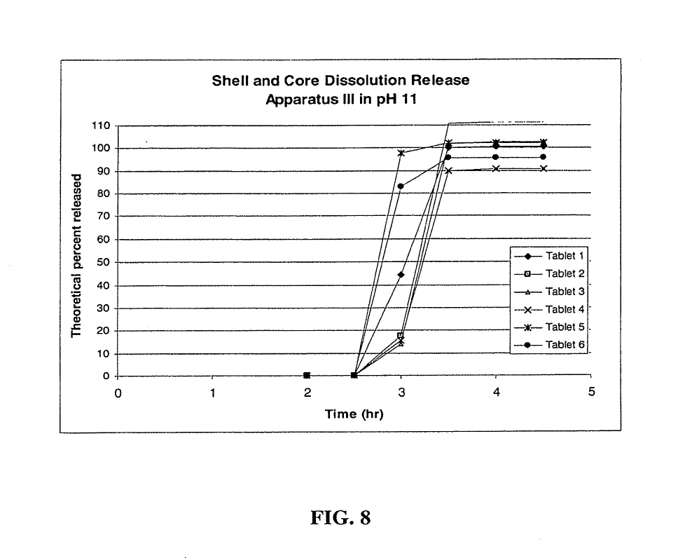

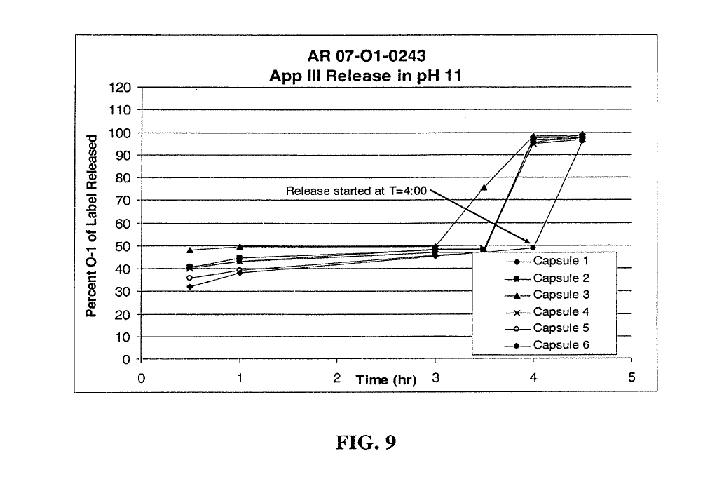

In vitro release was characterized by the use of a U.S. Pharmacopeia (USP) Apparatus III reciprocating cylinder. 250 mL of a pH 11 phosphate buffer at 37° C. was selected as the release medium because of omeprazole's stability at this pH. Results are shown in Table 3 and in FIG. 8.

TABLE 3
Representative Data from Shell and Core Dosage Form
Percent Release corrected for bead total content (%)

| Tablet | 2 hr | 2.5 hr | 3 hr | 3.5 hr | 4 hr | 4.5 hr |
|---|---|---|---|---|---|---|
| 1 | 0 | 0 | 44.5 | 100.0 | 100.5 | 100.5 |
| 2 | 0 | 0 | 17.8 | 110.7 | 111.3 | 111.3 |
| 3 | 0 | 0 | 13.9 | 101.8 | 102.6 | 102.6 |
| 4 | 0 | 0 | 15.5 | 89.7 | 90.7 | 90.7 |
| 5 | 0 | 0 | 97.7 | 102.4 | 102.4 | 102.4 |
| 6 | 0 | 0 | 82.7 | 95.5 | 95.5 | 95.5 |
| Average | 0 | 0 | 45.4 | 100.0 | 100.5 | 100.5 |
| Stdev | 0 | 0 | 36.82 | 7.07 | 7.00 | 7.00 |
| % RSD | 0 | 0 | 81.16 | 7.07 | 6.97 | 6.97 |

### Example 4

### Capsule Insert

In one embodiment, drug dosage forms that (1) are gastric retentive due to hydrated-state swelling, and (2) deliver multiple doses of an active pharmaceutical ingredient (API or drug), separated by a pharmacologically desirable time, in an immediate-release mode, from a single dosage form are provided. This example illustrates a dosage form as illustrated in FIGS. 4A-4E comprising at least two compression molded (or otherwise molded) modular plugs (called “inserts”) comprised of at least a swellable, erodible polymer (for example, polyethylene oxide) that are inserted into a commercially available, pharmaceutical capsule (for example, a gelatin capsule), along with at least one active pharmaceutical agent, for example, omeprazole. These dosage forms, when introduced into the stomach, initially swell and then erode over a pharmacologically desirable time (for instance, 3-5 hours) before releasing the drug in an immediate release fashion. The inserts in this illustrative embodiment are identical in shape and cylindrical, with one end having a deep cup (or pocket) with tapered walls, and the other end having a flat bottom and a taper of the same angle as that of the tapered walls on the other end of the insert. This shape allows the inserts to be “stacked”, while leaving a pocket between them for inclusion of the drug.

In this illustrative embodiment, the first pulse is designed to be released immediately after dosing. The drug, omeprazole, is added in the form of enterically protected coated sugar spheres, into the capsule outside of the inserts such that after the capsule dissolves, the first pulse is released. The second, or subsequent, dose of drug is inserted into the pocket created by the modular inserts. Upon introduction into the stomach, the gelatin capsule dissolves allowing the first pulse of drug to be released from the dosage form. Simultaneous with the dissolution of the gelatin capsule, the stacked polymeric inserts hydrate, swell, and as such, seal the joint between each insert, sealing the second pulse into the pocket between the inserts. The time of delay between the pulses can be controlled by varying the molecular weight of the polymer employed, and/or other well established formulation practices designed to extend erosion time. The example configuration provides a ˜1.4 mm minimum wall thickness from the inner chamber when the inserts are stacked to the outside wall of the inserts. It is the erosion through this thinnest part of the stacked insert assembly that provides the release of the second-pulse beads entrapped inside the insert chamber.

Multiple pulses of drug can be provided by adding multiple doses to one dosage form, and separating the pulses, both physically and temporally, by the addition of multiple molded inserts. Other dosage forms can deliver a drug in a typical, sustained-release mode, in addition to the pulsatile delivery, by incorporating drug into the insert along with the, swellable, erodible polymer. As the insert swells, the drug diffuses out, or is released as the polymer erodes, depending on the aqueous solubility of the drug.

This example describes inserts designed for manufacture on a typical rotary tablet press using a commonly available tooling type. In this example, the tooling was obtained through Natoli Engineering. Following the formulation insert screening described below, a suitable formulation was manufactured on a Piccola RLC 10-station rotary press (Riva Corp., Argentina). The use of a swellable, erodible polymer provides gastric retention and retards the release of the omeprazole containing, enteric coated beads. The inclusion of an excipient, such as a polyol, i.e. mannitol, promotes the catastrophic rupture, following an appropriate delay, of the shell to provide the IR burst of the beads. This teaching also applies to the illustrative shell and core dosage form described in Example 3. A lower MW polymer such as Polyox 1105 (MW=900,000 AMU), with a polyol such as Pearlitol 300 DC, and a lubricant such as magnesium stearate, USP (Mallinckrodt Corp. Hazelwood, Mo.), provides an acceptable delay and delivers the IR burst in the form of enteric coated omeprazole containing bead.

An exemplary capsule insert formulation is comprised of 70% Polyox 1105, 29.5% Pearlitol 300 DC, and 0.5% mg stearate.

In vitro release was characterized by a United States Pharmacopoeia (USP) Apparatus III dissolution tester. Release media was a pH 11 phosphate buffer at 37° C., chosen due to fact that omeprazole has been shown to be stable at pH 11. Results are shown in Table 4 below and in FIG. 9.

TABLE 4
Percent omeprazole of label released (%)

| | Percent omeprazole of label | | | | | |
|---|---|---|---|---|---|---|
| | released (%) at indicated time (hours) | | | | | |
| Dosage Form Test # | 0.5 | 1 | 3 | 3.5 | 4 | 4.5 |
| 1 | 32.0 | 37.9 | 45.2 | 46.9 | 95.4 | 99.0 |
| 2 | 40.3 | 44.5 | 47.8 | 47.8 | 96.7 | 97.0 |
| 3 | 47.8 | 49.5 | 49.5 | 75.6 | 98.3 | 98.3 |
| 4 | 39.9 | 43.2 | 48.5 | 48.5 | 95.0 | 96.7 |
| 5 | 35.6 | 39.3 | 45.9 | 46.9 | 97.3 | 97.7 |
| 6 | 40.6 | 43.2 | 46.9 | 46.9 | 48.8 | 96.0 |
| Average | 39.4 | 43.0 | 47.3 | 52.1 | 88.6 | 97.5 |
| Std. Dev. | 5.34 | 4.10 | 1.63 | 11.52 | 19.52 | 1.10 |
| % CV | 13.56 | 9.55 | 3.44 | 22.13 | 22.03 | 1.13 |

Upon dissolution testing, it was observed that some of the beads became stuck to each other and to the polymeric insert, which could slow the release of omeprazole from the dosage form. To ensure complete release of a 20 mg payload for each pulse within 30 minutes, a number of additives were examined. A small percentage (˜0.5-5%) of Talc, USP (Spectrum Chemicals, New Brunswick, N.J.) did not appear to improve dissolution and may have further retarded bead release, perhaps due to its hydrophobicity. Other excipients and additives that can improve dispersion of the beads upon liberation from the dosage form include Pearlitol, Polyplasdone XL, and the surfactant sodium lauryl sulphate (Spectrum Chemicals).

### Example 5

### Dry Polymer Bed Surrounding IR Core in Capsule

Drug dosage forms that provide a delayed pulse drug released by a core immediate release tablet containing acid-protected PPI placed into a dry polymer bed (such as of polyethylene oxide) which is in a capsule and wherein the bottom contains an insoluble polymer (such as ethylcellulose) are also provided. For example, the delayed pulse can be released by a core immediate release tablet containing acid-protected PPI placed into small cup placed in the bottom of a capsule (to receive the core and assure the core remains upright in the center of the capsule) and with the sides and top filled with a dry polymer bed (such as of polyethylene oxide).

Thus, the advantages of the core and shell embodiment of the dosage can be provided in capsule form. The capsule form also provides a convenient means to provide an immediate release pulse of drug in addition to the delayed pulse of drug release. In one illustrative embodiment, a core of the same formulation as the core and shell described in Example 1 is employed, but the core is shaped uniquely to fit inside a capsule body. For example, a cylindrical tablet is centered into a capsule body, and a dry-fill polymer bed of similar constitution as the shell of the core and shell surrounds the cylindrical core on all sides. As with the core and shell dosage form, the delay and gastric retention is derived from the swellable, erodible polymer matrix, but as the thickness of the polymer surround, in relation to the core, can be important for erosion timing, steps can be taken to ensure similar powder bed thickness on all sides of the core. In one embodiment to minimize this variation, half the core is surrounded with an insoluble matrix (a non-erodible polymer), leaving only the polymer half to erode, reducing delay-release time variation.

Testing demonstrated that dry fill POLYOX in capsules hydrates fast enough for the polymer to gel and promote gastric retention for a desired time period (2-6 hours). Release data was variable, however, with some capsules releasing core contents within 1 hour, others within the same lot releasing within 4 hours. ETHOCEL (ethylcellulose by Dow Chemicals) was examined as an insoluble surround but did not, when put filled into a capsule, remain together optimally during initial disintegration studies. Polymeric excipients, such as KLUCEL (hydroxyproply cellulose (HPC, Hercules, Welmington, POVIDONE by ISP), at high molecular weights were added at various weight percentages from 5% to 35%. An optimal blend consisted of 80% ETHOCEL STD 100, 15% POLYOX 303 Fine Particle, 5% POVIDONE, and remained intact for a suitable amount of time. PoOLYOX remains intact at the POLYOX/ETHOCEL blend junction, while non-POLYOX based ETHOCEL blends showed a tendency to split at that junction immediately prior to capsule-body dissolution.

An additional first pulse was added to the very top of this capsule to deliver two pulses. The first pulse blend consisted of XYLITAB and beads to prevent sticking to the polymer bed or to one another. Two pulses were delivered from these dosage forms, separated by ˜2-4 hours. It is desirable for such capsules to be completely full to avoid the shifting of capsule contents, which could create undesirable voids around the core.

### Example 6

### Manufacturing Processes

A study was to evaluate materials and process conditions for a fluidized bed film-coating process for particles with various batch sizes (0.7-1.8 kg) and two different spraying dispersions: 20% Opadry II Blue (sub-film for placebo or test use only) and 20% AcrylEZE MP (enteric film). No active pharmaceutical ingredient was used for this work. Fluidized bed coating of particles involves repetitive movement of core particles through an atomized spray region in a relatively controlled manner. Each cycle of movement involves wetting followed by drying cycle. The balance of these cycles provides the appropriate quality and consistency in the product. An understanding of the parameter relationships provides a predictive tool for film-coating processes.

In this example, the fluidized bed filth-coating process was performed on Vector FL-M-1 Fluid Bed with Würster partition. Würster partition enhanced the particle movement within the bed. The spray nozzle was placed at the bottom centre of the distributor plate so that the movement of coated particles was in the same direction as the fluidized gas. The placebo bead manufacturing process conditions were used in manufacturing active bead products, as also described in this example. The equipment used for placebo bead testing and manufacturing included the following: Vector FL-M-I, Barnant Mixer, Watson Marlow 505 DU/RL Pump, Mettler Balances, HR 73 Halogen Moisture Analyzer, Leica Microscope, W.S. Tyler Vibratory Sieve Shaker, and Vankel Tap Density Tester.

Initially, the core was selected. The core is ideally spherical in shape and has a smooth surface to ensure good flowability. The shape and the surface of the sugar sphere can be evaluated visually using a microscope. Moisture level is important factor in evaluation of microbial growth accessibility of the sugar spheres. Moisture level of sugar beads can be evaluated by determining the LOD with HR 73 Halogen Moisture Analyzer. Bulk and tap densities can be determined for information purposes as follows. A graduated cylinder is filled with a certain amount of material (82-88 g), and the volume recorded to determine the material bulk density. Tap density can be determined with a help of a Tap Density Tester by exposing the material to 100 taps per test and recording the new volume. Sugar particle size distribution is ideally in a narrow range to ensure uniform application of coating material and can be evaluated by a sieving technique. For example, a 100 g material sample can be sieved for five minutes on Vibratory Sieve Shaker and the fractions are weighed on Mettler balance to estimate size distribution. After evaluations such as those described above, the sugar core or sphere selected was NP Pharm SUGLETS® (NP Pharm, Product Code PF008, Lot No. 606C, bead size 600/710 μm). Other sugar spheres evaluated (Paulaur), had a wider size distribution range and were less spherical and smooth. The LOD and bulk and tap density values for the spheres from both manufacturers (NP Pharm and Paulaur) were comparable, although, for the 300/425 μm sizes, moisture content appears higher for the Paulaur spheres.

Spray process development work on a Vector Fluid Bed FL-M-1 was performed with two types of spray dispersions (20% Opadry II Blue and 20% AcrylEZE MP) and two Würster partition sizes: 6″ and 8″ (for different batch sizes). The goal for this development work was to establish film-coating process at low product temperatures of 35±2° C. while minimizing the process time by using high spray rates. The development work was focused on evaluating the quality of the fluidized bed at various air flow levels and different spray rates while maintaining the constant product temperature.

The excipient information and formula for the Opadry II Blue spray dispersion was Opadry II Blue (Colorcon, Product Code Y-22-10564, Lot No. WP612148, in an amount of 20% w/w) and purified water, USP (Ricca Chemical Co., Product Code 9190-5, Lot No. 1508075/1408632, in an amount of 80% w/w). The procedure for preparation of Opadry II Blue dispersion is as follows. The water is placed into a mixing vessel and stirred to form a vortex without drawing air into the liquid with the impeller being in the center as close to the bottom of the vessel as possible; then, the Opadry II Blue powder is added to the vortex, avoiding powder flotation on the liquid surfacem and mixed for approximately 60 minutes. Although the manufacturer of Opadry II Blue (Colorcon) has recommended working temperature of ≦40° C. for similar spray processes, a low product temperature of 35±2° C. was selected due to the temperature sensitivity of the active ingredient (omeprazole) to be used in manufacturing of the active bead material. A low temperature of 35±2° C. was selected to ensure product stability.

The factors used to identify optimal film-coating process conditions were: good fluidized bed flow; no build-up of bead material on the equipment interior (Würster partition, exhaust filter or vessel sides); and visual inspection under microscope on samples taken throughout the process to ensure no agglomerates (including small, two or three sphere agglomerates) and good color uniformity of the film (Opadry II Blue provides a good contrast to the white sugar core) as an indicator of uniform coating.

The 20% Opadry II Blue dispersion, contained in a stainless steal beaker, was gently agitated during the spraying process. The beaker was placed on Mettler SG 8001 Balance in order to monitor the spray rate change over time. A Watson Marlow 505 DU/RL pump was used to control the flow of the dispersion into a Vector FL-M-1 Fluid Bed system.

The critical coating parameters were evaluated during the manufacture of nine placebo lots. Broad parameter ranges were examined to determine the optimal process conditions based on the above described criteria. Some of the parameter values were kept constant during the development work based on the defined application or recommendation from the equipment manufacturer. The coating parameter ranges evaluated during coating process development work with 20% Opadry II Blue were: (1) Würster Partition Elevation, range 0.125-0.5″ (6″ Würster) and 0.75-1″ (8″ Würster); (ii) Spray Rate, range 4-12 g/min; (iii) Air Flow, range 45-60 CFM; and (iv) Batch Size (at start of coating process step, range 0.7-1.5 kg (6″ Würster) and 1.8 kg (8″ Würster). The parameters kept constant during coating process development work with 20% Opadry II Blue were Inlet Air Temperature 52±2° C., Product Temperature 35±2° C., Nozzle Air Pressure 32 psi, Accelerator Air Pressure 30 psi, Mixer setting 2.0, Nozzle extension and spacer 1/16″, and Teflon Distribution plate 100FP.

Key observations from the coating work with Opadry II Blue were as follows: formation of a good quality fluidized bed is compromised when the Würster partition is elevated at 0.125-0.25″; optimal Würster elevation is in the range 0.375-0.5″ (6″ Würster) and 0.75-1″ (8″ Würster); a good balance of the wetting/drying cycle of the fluidized bed can be achieved when the spray rates are ≦10 g/min for batches of 0.7-1.3 kg and ≦12 g/min for batches of 1.3-1.8 kg; nozzle air pressure of 32 psi provides good quality spray pattern for this application; material build up on the exhaust filter occurs for airflow values above 50CFM. The above described conditions provide uniform bead coating as detected from the visual examination under microscope of samples taken at different time points throughout the process.

The AcrylEZE MP enteric coat was composed of the following: AcrylEZE MP (Colorcon, Product Code 93018508, Lot No. WP603787, in an amount of 20% w/w); 30% Simethicone Emulsion, USP Dow Corning, Product Code 3125424, Lot No. 0002410491, in an amount of 0.1% w/w); and purified water, USP (Ricca, as above, in an amount of 79.9% w/w). The procedure for preparing the AcrylEZE MP dispersion is as follows. The 30% Simethicone Emulsion is placed into a mixing vessel, and water is added and stirred to form a vortex without drawing air into the liquid with the impeller being in the center as close to the bottom of the vessel as possible. The AcrylEZE MP powder is added to the vortex, avoiding powder flotation on the liquid surface, and mixed for approximately 60 minutes. The dispersion mixture is passed through a 250 μm sieve prior to the coating process. The 20% AcrylEZE MP dispersion, contained in a stainless steal beaker, was gently agitated during the spraying process. The beaker was placed on Mettler SG 8001 Balance in order to monitor the spray rate change over time. A Watson Marlow 505 DU/RL Pump was used to control the flow of the dispersion into the Vector FL-M-1 Fluid Bed system.

The quality criteria used for this film-coating process is identical to the one defined for the Opadry II Blue coating process. Critical process parameters were evaluated during nine placebo runs. Higher product temperatures (35-40° C.) were used in the early stage of this development work. The product temperature was later changed to 30±2° C. as AcrylEZE material appears stickier at elevated temperatures. Spray rates of >7 g/min (used in the earlier development work) appeared to cause agglomeration. Once the spray rates were adjusted to values of 5-7 g/min, the overall quality of the process significantly improved. A build up of AcrylEZE material on the tip of the spray nozzle occurred when the nozzle pressure was kept at 32 psi but did not occur when the nozzle pressure was adjusted to 36 psi.

Coating parameter ranges evaluated during coating process development work with 20% AcrylEZE MP were: Spray Rate, range 5-14 g/min; Air Flow, range 40-70 CFM; Nozzle Air Pressure, range 32-36 psi; Inlet Air Temperature, range 40-59° C.; Product Temperature, range 30±2° C. 40±2° C.; and Batch Size (at start of coating process step), range 0.7-1.3 kg (6″ Würster) and 1.3-1.4 kg (8″ Würster). Parameters kept constant during coating process development work with 20% AcrylEZE MP were: Würster Partition Elevation, 0.375-0.5″ (6″ Würster) and 1″ (8″ Würster); Accelerator Air Pressure, 30 psi; Mixer setting 2.0; Nozzle extension and spacer, 1/16″ Teflon; and Distribution plate, 100FP. Key observations from the coating work with AcrylEZE MP were as follows: the optimal Würster elevation is in the range 0.375-0.5″ (6″ Würster) and 0.75-1″ (8″ Würster); a good balance of the wetting/drying cycle of the fluidized bed can be achieved when the spray rates are 55 g/min for batches 0.7-1.3 kg and 57 g/min for batches 1.3-1.8 kg; nozzle air pressure of 36 psi provides good quality spray pattern for this application; and airflow above 50CFM causes build up of material on the exhaust filter.

This development work showed that the manufacturing process parameters for placebo coated sugar spheres on Vector Fluid Bed FL-M-1 depends primarily on batch size and type of coating dispersion. The batch size determines the Würster partition (6″ or 8″); Würster partition elevation; and spray rate. The type of coating dispersion determines the process values for inlet temperature, nozzle air pressure, and spray rate. Critical parameters for the spray coating process were determined to be the Wüster partition elevation, spray rate, air flow, and inlet air temperature. The parameters used for development of process conditions for active bead manufacturing were as follows. For the 20% Opadry II Blue process, the Würster partition size (″) was 6 for batch size 0.7-1.3 kg and 8 for batch size 1.3-1.8 kg; the Würster partition elevation (″) was 0.375-0.5 for batch size 0.7-1.3 kg and 0.75-1 for batch size 1.3-1.8 kg; the inlet air temperature was 15±2° C. above desired product temperature; the air flow (CFM) was 50; the nozzle air pressure (psi) was 32; and the maximum spray rate (g/min) was 10±1 for batch size 0.7-1.3 kg and 12±1 for batch size 1.3-1.8 kg.

For the 20% AcrylEZE MP process, the Würster partition size (″) was 6 for batch size 0.7-1.3 kg and 8 for batch size 1.3-1.8 kg; the Würster partition elevation (″) was 0.375-0.5 for batch size 0.7-1.3 kg and 0.75-I for batch size 1.3-1.8 kg; the inlet air temperature was 10±2° C. above desired product temperature; the air flow (CFM) was 50; the nozzle air pressure (psi) was 36; and the maximum spray rate (g/min) was 5±1 for batch size 0.7-1.3 kg and 7±1 for batch size 1.3-1.8 kg.

Two active bead batches with a design (from interior to exterior) as follows: bead core of sugar spheres of size 600-710 microns; active coat of omeprazole (20-40% weight gain); sub-coat of Opadry (3-5% weight gain); enteric coat of AcrylEZE (25-40% weight gain). The beads were with tight active agent content range (STD<1%) and with desired acid resistance characteristics. All above batches were prepared in <2 kg runs on a Vector FL-M-1. Beads with 355/425 μm sugar cores can be made on the same equipment and with similar bead formulation. The smaller size beads are intended for the capsule with insert design as they fit well the space in the inserts.

Bead manufacturing in a fluid bed system can also be conducted using beads that contain a microcrystalline cellulose (MCC) core (Celphere CP 305 and Celphere CP 507). Opadry® coat is applied on these beads on top of the active omeprazole coat. Batch sizes up to 6 kg can be prepared on a Vector FL-M-15 Fluid Bed system (process run at Vector Corporation).

Particle manufacturing with extrusion (MCC based core) can be used to manufacture omeprazole particles with size of 0.5 nun and 0.7 mm by using an extrusion process (Emerson Resources, Inc., using a dome extruder from LCI, model DG-L-1, and a 230 mm spheronizer equipped with a 2 mm plate). The first step of the process was extrusion of particles that contained 35-50% ompeprazole and the remainder MCC. In other runs, these particles were directly coated with enteric coat (EUDRAGIT L30D55 polymer) that contained Triethyl Citrate (TEC) as a plasticizer (2-10% in the final coat). The coating process was done on a Vector FL-M-1. These particles showed very uniform drug content (STD≦1%) and had the desired acid resistance characteristics.

Dosage forms having a core and shell configuration can be manufactured on a rotary tablet press (Manesty Betapress) working with 1 kg batch sizes. Active formulations with bead amount in the blend of 20-60% have been prepared. Active agent uniformity increases with increased bead content in the core tablet (STD % 1-5; 1% achieved for the 60% bead core formula). Core and shell manufacturing can also be conducted at contract manufacturers (Patheon/MOVA®) based on the guidance provided herein. In one illustrative embodiment, the core is composed of the following ingredients, within the ranges shown parenthetically (excipients may be used “as is” or with granulation by conventional pharmaceutical granulation processes or in any combination thereof): sugar-starch spheres (25% of core); polyethylene oxide (10-20%); polyethylene glycol (15-35%); POVIDONE (polyvinyl pyrrolidone, 3-6%); croscarmellose sodium (3-5%); sodium starch glycolate (2-5%); CROSPOVIDONE (cross-linked polyvinyl pyrrolidone, 3-15%); microcrystalline cellulose—fine particle (5-25%); microcrystalline cellulose—coarse particle (10-20%); pre-gelatinized starch (15-40%); magnesium stearate (0.5-2%); and talc (0.5-4%).

In one illustrative embodiment, the shell is composed of the following ingredients, within the ranges shown parenthetically (excipients may be used “as is” or with granulation by conventional pharmaceutical granulation processes or in any combination thereof): XYLITAB® xylitol (20-30% of shell); poly(ethylene oxide) (typically type 1105 with a molecular weight of about 900,000, determined rheologically, 70-80%); polyethylene glycol (up to 10-20%); cross-linked polyvinyl pyrrolidone (up to 10-20%); microcrystalline cellulose (up to 10-20%); magnesium stearate (about 1%); and optionally binders such as poly(vinyl pyrrolidone) (POVIDONE), cross-linked polyvinyl pyrrolidone (COPOVIDONE), hydroxypropyl methylcellulose and the like (3-8%).

Dosage forms as depicted in FIGS. 5A-5B can be can be manufactured on tableting equipment from Kikusui.

### Example 7

### Other Embodiments

In one embodiment, the delayed pulse is created by a core immediate release tablet containing acid-protected PPI placed into a dry polymer bed (such as of polyethylene oxide (PEO)) which is in a capsule.

In one embodiment, the delayed pulse is created by placing acid protected granules, pellets, or beads placed into a cup which fits snugly into a capsule, and then the top is sealed with poly(ethylene oxide) (PEO) either as PEO powder which is tamped or is a pre-made (via compression or injection molding) PEO plug or cap, and then the capsule top (not enteric coated) is placed on the capsule bottom to seal the capsule

In one embodiment, the delayed pulse is created by a core tablet with an erodible coating which releases the acid-protected PPI in a pulse, wherein the acid-protected PPI can be enteric or delayed release coated particle, bead or pellet, or alternatively, a particle bead or pellet containing base, wherein the coating is applied by conventional pan-coating techniques, to create a type of “shell and core” tablet.

In one embodiment, the delayed pulse is created by a core tablet with an erodible coating which releases the acid-protected PPI in a pulse, wherein the acid-protected PPI can be enteric or delayed release coated particle, bead or pellet, or alternatively, a particle bead or pellet containing base, wherein the coating is applied by powder layering.

In one embodiment, the delayed pulse is created by a core immediate release tablet containing acid-protected PPI surrounded by polymer (along with fillers and other excipients as necessary) extruded as two sheets sandwiching the tablet and the edges are sealed. The tablet core is made separately and placed between two ribbons of extruded, swellable, erodible polymer. A sealing/cutting machine would be used to finish the dosage form.

In one embodiment, the delayed pulse is created by a core immediate release tablet containing acid-protected PPI placed in a capsule made of PEO by compression molding or injection molding and sealed by the addition of a capsule top or compressing (with or without heat) the edges of the top to seal the capsule.

In one embodiment, the delayed pulse is created by a core immediate release tablet containing acid-protected PPI placed in a “half tablet” cup like a clam shell with a protruding lip for sealing and then a second “half tablet” cup is placed on top, and the two are sealed together by compression around the lip edges only (core tablet does not undergo two compression cycles) with or without heat. In a related embodiment, the first half of the clam shell is sealed with a flat sheet and then attached to a second half-tablet which contains acid protected PPI wherein the second half erodes much more quickly than the first half. The “half tablet” cup can be manufactured using typical (or slightly modified) tablet compression tooling. Two cups can be placed together with a core inside, then, another tool could compress the perimeter to seal the two cups together.

In one embodiment, the delayed pulse is created by acid protected granules, pellets or beads placed into an enteric bottom which has been coated with an enteric coating. Then, a PEO plug of PEO dry powder is placed over the bottom, and IR beads are added on top. Then, the capsule top (not enteric coated) is placed on the capsule bottom.

In one embodiment, the delayed pulse is created by acid protected granules, pellets or beads placed into an enteric bottom which has been coated with an enteric coating. Then, a PEO plug of compressed PEO is placed over the bottom, IR beads are added on top, and the capsule top (not enteric coated) is placed on the capsule bottom to seal the capsule.

In one embodiment, the delayed pulse is created by placing acid protected granules, pellets or beads of acid-protected PPI placed into a polymer matrix also containing granules, pellets or beads which are enteric coated and contain disintegrant and/or other excipients such that the dissolution of the enteric coating of the disintegrants leads to catastrophic failure of the matrix wherein the matrix may be either a tablet or one layer of a bilayer tablet.

In one embodiment, the delayed pulse is created by a combination of multiple (two or more) pellets containing acid-protected PPI that are coated with PEO or other polymer (via powder layering or other technique) and the immediate release is created by multiple (two or more) pellets containing disintegrant, with both types of pellets placed in the same capsule.

In one embodiment, a dual release dosage form suitable for acid stable drugs is provided by coating the exterior of a gastric-retentive dosage form of the drug with a layer of drug admixed with suitable excipients for rapid erosion.

In one embodiment, a dual release (initial plus delay pulse drug release) dosage form is provided by placing a gastric-retentive core and shell finished tablet containing the drug into a hopper-fed core and shell machine onto which an additional drug-containing layer is applied, as in the case of a bilayer tablet above, wherein one half of the tablet is a core and shell, and the other half is a compressed-on matrix of drug containing particles, including, in one embodiment, enteric-coated PPIs.

In one embodiment, a dual release dosage form is provided by placing a core and shell tablet inside a capsule, into which another drug containing unit, i.e., enteric-coated beads, is added. The capsule is then sealed and contains a tablet to provide delayed release and beads to deliver the initial pulse.

1.-78. (canceled)

79. A dosage form comprising a first dose of drug that is released from the dosage form substantially immediately after oral administration, and a second dose of drug that is released from the dosage form substantially after oral administration,
wherein the dosage form comprises a layer comprising a swellable erodible hydrophilic polymer that swells unrestrained dimensionally in water, and
a tablet core which comprises the second dose of drug, and
wherein the tablet core is encased by the layer comprising the swellable erodible hydrophilic polymer.

80. The dosage form according to claim 79, wherein the tablet core comprises a plurality of beads, wherein the plurality of beads comprises the second dose of drug.

81. The dosage form according to claim 79, wherein each of the plurality of beads comprises a bead core and a drug layer surrounding the bead core, wherein the drug layers of the plurality of beads comprise the second dose of drug and optionally a pharmaceutically acceptable excipient.

82. The dosage form according to claim 81, wherein each of the plurality of beads comprises a sub-coat layer between the bead core and the drug layer.

83. The dosage form according to claim 80, wherein each of the plurality of beads is coated with a protective coating, wherein the protective coating is an enteric coating, a coating that erodes at a controlled rate, or a stabilizing component.

84. The dosage form according to claim 81, wherein the bead core comprises a starch, a sugar, or microcrystalline cellulose.

85. The dosage form according to claim 80, wherein the tablet core comprises the plurality of beads which are dispersed in a matrix, wherein the matrix is comprised of one or more pharmaceutically acceptable excipients.

86. The dosage form according to claim 79, wherein the tablet core comprises the second dose of drug and one or more excipients compressed together to form the tablet core.

87. The dosage form according to claim 81, wherein the one or more pharmaceutically acceptable excipients include polyethylene glycol or polyethylene oxide in combination with a polyol, a sugar or a diluent.

88. The dosage form according to claim 79, wherein the swellable erodible hydrophilic polymer is poly(ethylene oxide), hydroxypropyl methyl cellulose, or a combination of poly(ethylene oxide) and hydroxypropyl methyl cellulose.

89. The dosage form according to claim 79, wherein the layer encasing the tablet core further comprises a polyol, lactose, mannitol, or polyplasdone.

90. The dosage form according to claim 79, wherein the layer encasing the tablet core comprises poly(ethylene oxide) and mannitol.

91. The dosage form according to claim 79, wherein the swellable erodible hydrophilic polymer has a molecular weight ranging from 9×10⁵ Daltons to 8×10⁶ Daltons or has a viscosity of a 1% water solution of the polymer at 25° C. ranging from 4500 to 7500 centipoise.

92. The dosage form according to claim 79, wherein the layer encasing the tablet core comprises about 70% poly(ethylene oxide) having a molecular weight of 900,000 Daltons and 29.5% mannitol.
